(19) United States
(12) Patent Application Publication
(10) Pub. No.: US 2024/0277710 A1
(43) Pub. Date: Aug. 22, 2024

(54) PHARMACEUTICAL COMPOSITION FOR TREATMENT OF CANCER AND USE THEREOF

(71) Applicant: HINOVA PHARMACEUTICALS INC., Chengdu, Sichuan (CN)

(72) Inventors: Jing LI, Chengdu, Sichuan (CN); Wu DU, Chengdu, Sichuan (CN); Yongxu HUO, Chengdu, Sichuan (CN); Xinghai LI, Chengdu, Sichuan (CN)

(21) Appl. No.: 18/563,987

(22) PCT Filed: May 24, 2022
(86) PCT No.: PCT/CN2022/094725

§ 371 Date: Nov. 24, 2023

(30) Foreign Application Priority Data
May 25, 2021 (CN) 202110572563.8

(51) Int. Cl. A61K 31/506; A61P 35/00

(52) U.S. Cl. CPC A61K 31/506; A61P 35/00

(57) ABSTRACT

A pharmaceutical composition for the treatment of cancer and a use thereof are provided. The pharmaceutical composition consists of an FAK inhibitor and a CD44 inhibitor. Also provided is a drug combination of an FAK inhibitor and a CD44 inhibitor for the treatment of cancer. The CD44 inhibitor and FAK inhibitor drug combination can have synergistic effects, significantly improving tumor inhibition effects, reducing toxic side effects, and overcoming tumor drug resistance.

### TECHNICAL FIELD

The present invention belongs to the field of medicine, and specifically relates to a pharmaceutical composition for treating cancers and its use.

### BACKGROUND TECHNOLOGY

Cancer usually refers to all malignant tumors, with biological characteristics such as abnormal cell differentiation and proliferation, uncontrolled growth, infiltration, and metastasis. Every year, there are many deaths due to various types of cancers, posing a serious threat to human survival. At present, the clinical treatment of tumors is still mainly based on surgery and chemotherapeutical drugs. However, since entering the 21st century, molecular targeted therapy is playing an increasingly important role. Compared to traditional cytotoxic medicaments, molecular targeted drugs have become a hot topic in the research and development of anti-tumor medicaments, since they target the characteristics of tumor cells that are different from normal cells, change blind attack to shooting the target, and thus reduce toxic side effects on normal organs and tissues, but play strong anti-tumor effects, thereby improving the life quality of patients.

CD44 is a transmembrane glycoprotein of cell surface, mainly involved in heterogeneous adhesion, which refers to the adhesion of tumor cells to host cells and host matrix. Heterogeneous adhesion plays a promoting role in the invasion and metastasis of tumor cells. The literature “Anti-CD44 antibodies inhibit both mTORC1 and mTORC2: a new rationale supporting CD44-induced AML differentiation therapy” (Merzaban, J, S, et al. Leukemia: Official journal of the Leukemia Society of America, Leukemia Research Fund, U.K, 2016) has indicated that inhibiting CD44 with antibodies can lead to a decrease in the expression of two main pathways involved in abnormal growth of cancer cells: the PI3K (phosphatidylinositol 3-kinase) pathway and the mTOR (mammalian rapamycin target protein) pathway. Therefore, CD44 inhibitors have the potential to be applied in the treatment of cancer. Although CD44 inhibitors exhibit good anticancer activity, they have been found to have high toxic side effects in clinical trials (References “CD44V6-targeted imaging of head and neck squamous cell carcinoma:antivody-based approaches” and “Hyaluronic acid targeting of CD44 for cancer therapy: from receptor biology to nanomedicine”).

Focal adhesion kinase (FAK) is a non-receptor tyrosine protein kinase, which is an important cytoskeletal protein and key molecule in various signaling pathways in cells. FAK plays an important role in various stages of tumor occurrence, development, migration, and invasion. At present, FAK is regarded as a potential target for tumor treatment, and FAK inhibitors serve as ligands that can competitively bind to the binding site of FAK receptors with ATP, blocking the transmission of FAK-mediated growth and proliferation signaling pathways, leading to inhibition of malignant tumor cell growth and proliferation, and even leading to cell death at high doses. However, FAK inhibitors also have high toxic side effects. Moreover, with further research, it has been found that the biological role of FAK in the development of cancer conditions is complex, and its therapeutic effect on cancer cannot be determined.

In addition, drug resistance has always been a major challenge for the treatment of cancer. How to overcome the drug resistance is also a challenge in the study on cancer treatment. At present, there is no evidence of manufacturing a medicament for treating cancer by combining CD44 inhibitors and FAK inhibitors. Thereby, further research is needed to determine whether they have therapeutic effects on cancer, whether their toxic side effects are reduced, and whether they can overcome the drug resistance of cancer cells.

### SUMMARY OF THE INVENTION

The present invention is to provide a pharmaceutical composition for treating cancer and its use.

The present invention provided a pharmaceutical composition for treating cancer, which is composed of FAK inhibitors and CD44 inhibitors.

Further, the weight ratio of FAK inhibitor and CD44 inhibitor is 1:10-10:1.

Further, the FAK inhibitors are the following compounds, or optical isomers thereof, or tautomers thereof, or salts thereof, or prodrugs thereof, or hydrates thereof, or solvates thereof, and the following compounds include: Defactinib, CEP-28122, CEP-37440, TAE226, PF-562271, PF-431396, VS-4718, PF-573228, BI853520, IN10018, and APG-2449;

alternatively, the FAK inhibitor is a compound represented by formula I, or optical isomers thereof, or tautomers thereof, or salts thereof, or prodrugs thereof, or hydrates thereof, or solvates thereof,

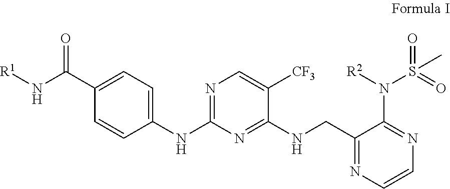

wherein, R¹ and R² are each independently selected from the group consisting of hydrogen, methyl, and trideuteromethyl;

alternatively, the FAK inhibitor is the following compounds, or optical isomers thereof, or tautomers thereof, or salts thereof, or prodrugs thereof, or hydrates thereof, or solvates thereof, and the following compounds are selected from the group consisting of:

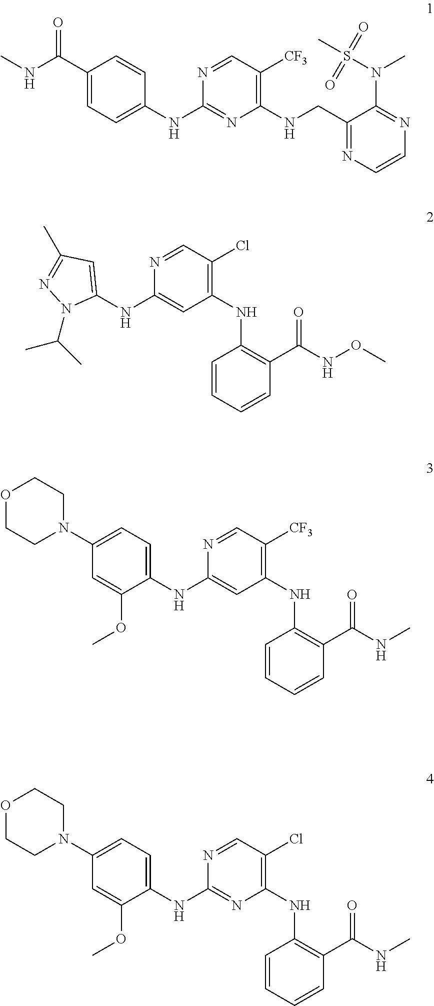

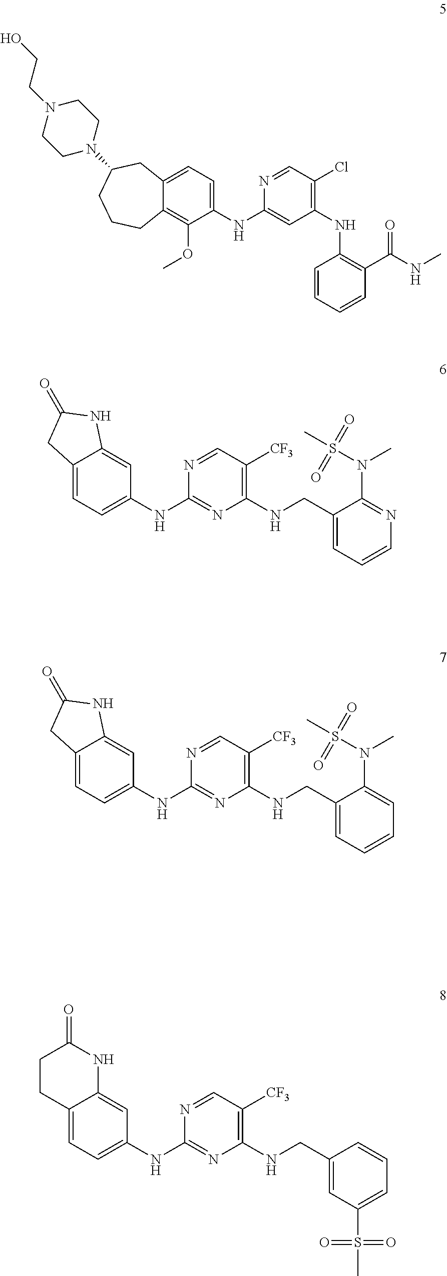 [SMILES: CNC(=O)c1ccccc1Nc1cc(Nc2ccc3c(c2OC)CCC[C@H](N2CCN(CCO)CC2)C3)ncc1Cl ; CN(c1ncccc1CNc1nc(Nc2ccc3c(c2)NC(=O)C3)ncc1C(F)(F)F)S(C)(=O)=O ; CN(c1ccccc1CNc1nc(Nc2ccc3c(c2)NC(=O)C3)ncc1C(F)(F)F)S(C)(=O)=O ; CS(=O)(=O)c1cccc(CNc2nc(Nc3ccc4c(c3)NC(=O)CC4)ncc2C(F)(F)F)c1]

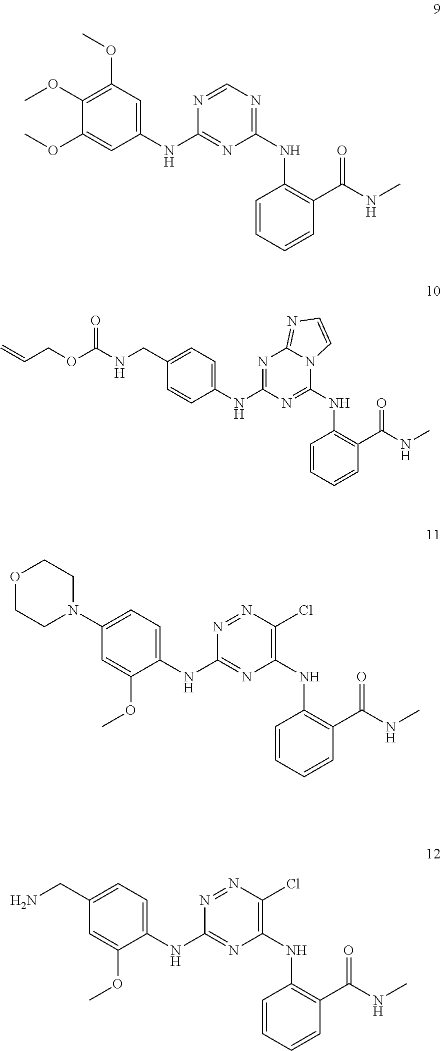

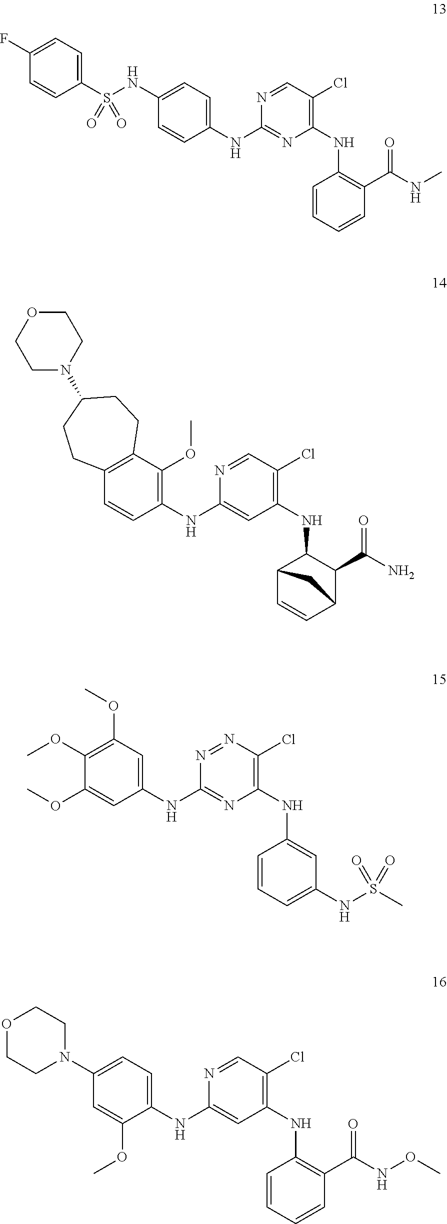

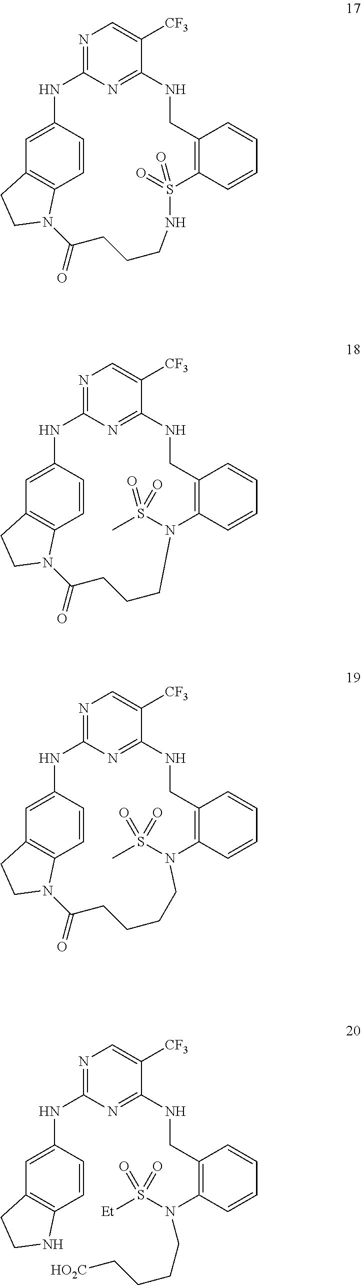 [SMILES: O=C1CCCNS(=O)(=O)c2ccccc2CNc2nc(ncc2C(F)(F)F)Nc2ccc3c(c2)CCN13 ; CS(=O)(=O)N1CCCC(=O)N2CCc3cc(ccc32)Nc2ncc(C(F)(F)F)c(n2)NCc2ccccc21 ; CS(=O)(=O)N1CCCCC(=O)N2CCc3cc(ccc32)Nc2ncc(C(F)(F)F)c(n2)NCc2ccccc21 ; CCS(=O)(=O)N(CCCCC(=O)O)c1ccccc1CNc1nc(Nc2ccc3c(c2)CCN3)ncc1C(F)(F)F]

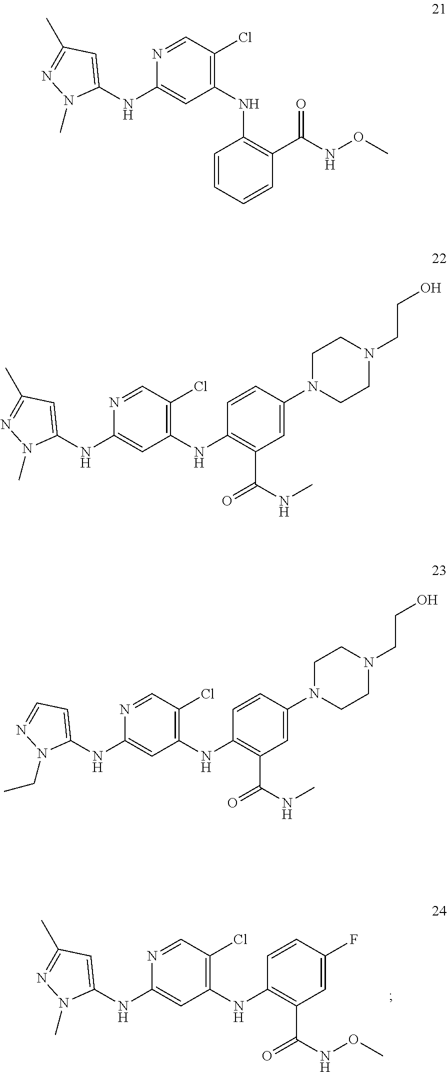

preferably, the FAK inhibitor is the following compounds, or optical isomers thereof, or tautomers thereof, or salts thereof, or prodrugs thereof, or hydrates thereof, or solvates thereof, and the following compounds include

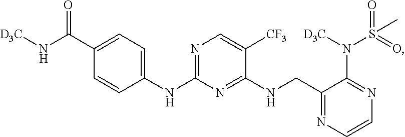 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]

### Defactinib or IN10018.

Further, the CD44 inhibitor is the following compounds, or optical isomers thereof, or tautomers thereof, or salts thereof, or prodrugs thereof, or hydrates thereof, or solvates thereof, or peptides, or antibodies, and the following compounds or peptides or antibodies include Angstrom6, PEP-1, AMC303, Anti-CD44 mAb H4C4, anti-CD44 mAb HI44a, anti-CD44 mAb IM7, anti-CD44 mAb KM201, RO5429083, RG7356, anti-CD44V6 mAb 2F10, anti-CD44V6 mAb U36, anti-CD44V6 mAb V6B3, anti-CD44V6 mAb VFF18, anti-CD44V6 mAb VFF4, anti-CD44V6, and mAb VFF7;

preferably, the CD44 inhibitor is the following compound, or optical isomers thereof, or tautomers thereof, or salts thereof, or prodrugs thereof, or hydrates thereof, or solvates thereof, and the following compound includes AMC303.

Further, the molar ratio of FAK inhibitor and CD44 inhibitor is 1:82-82:1;

preferably, the molar ratio of FAK inhibitor and CD44 inhibitor is 0.4-81:1.

Further, the FAK inhibitor is compound

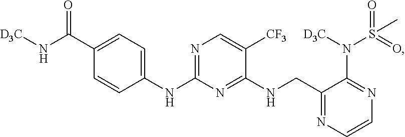 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]

or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-81:1;

alternatively, the FAK inhibitor is Defactinib, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-27:1;

alternatively, the FAK inhibitor is IN10018, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 1:1.

The present invention also provides a preparation method for a pharmaceutical composition mentioned above, which comprises the following steps: FAK inhibitors and CD44 inhibitors were weighed according to the pre-determined weight ratio, and then well mixed.

The present invention also provides the use of the above pharmaceutical composition in the manufacturer of medicaments for treating cancer;

preferably, the cancer is solid tumor, mesothelioma, melanoma, prostate cancer, breast cancer, glioblastoma, and brain cancer;

the solid tumors include mesothelioma, pancreatic cancer, soft tissue tumor, metastatic tumor, non-solid cancer, sarcoma, adenocarcinoma, lung cancer, breast cancer, lymphoma, gastrointestinal cancer, urogenital system cancer, prostate cancer, ovarian cancer; the gastrointestinal cancer includes colon cancer, and the urogenital system cancer includes renal, urothelial, or testicular tumors; the ovarian cancer includes advanced ovarian cancer;

the mesothelioma includes neurofibroma, renal cancer, lung cancer, small cell lung cancer, non-small cell lung cancer, KRAS-mutant non-small cell lung cancer, liver cancer, thyroid cancer, breast cancer, nervous system tumor, neurilemmoma, meningioma, neuroma, adenoid cystic carcinoma, ependymoma, ependymal tumor, malignant pleural tumor, malignant pleural mesothelioma, triplet tumor, negative breast cancer, non-blood malignant tumor, melanoma, colorectal cancer, leukemia, adenocarcinoma, and solid tumors;

the melanoma includes locally advanced melanoma, locally mutant N-Ras driven melanoma, and metastatic malignant skin melanoma; the colorectal cancer includes metastatic colorectal cancer; the leukemia includes acute myelogenous leukemia; the adenocarcinoma includes adenocarcinoma; the solid tumors include locally advanced solid tumors, metastatic solid tumors, and hepatocellular carcinoma;

the prostate cancer includes castration-resistant prostate cancer and metastatic castration resistant prostate cancer;

the brain cancer includes neuroepithelial tissue tumors, cerebral gliomas, astrocytomas, oligodendrogliomas, ependymal and choroidal plexus tumors, pineal tumors, neurocellular tumors, gangliocytoma, neuroblastoma, poorly differentiated tumors, embryonal tumors, glioblastoma multiform, medulloblastoma, schwannoma, meningioma, malignant lymphoma, cerebrovascular tumors, vascular malformations, capillary telangiectases, pituitary tumors, and metastatic tumors.

The present invention also provides a pharmaceutical preparation for treating cancers, which is made from the above pharmaceutical compositions, as active ingredients, in combination with pharmaceutically acceptable excipients or auxiliary ingredients.

The present invention also provides a drug combination for treating cancers, which comprises FAK inhibitors and CD44 inhibitors in the same or different specifications administered simultaneously or separately, as well as pharmaceutically acceptable carriers;

preferably, the weight ratio of FAK inhibitor and CD44 inhibitor is 1:10-10:1;

alternatively, the molar ratio of FAK inhibitor and CD44 inhibitor is 1:82-82:1;

more preferably, the molar ratio of FAK inhibitor and CD44 inhibitor is 0.4-81:1.

Further, the FAK inhibitors are the following compounds, or optical isomers thereof, or tautomers thereof, or salts thereof, or prodrugs thereof, or hydrates thereof, or solvates thereof, and the following compounds include: Defactinib, CEP-28122, CEP-37440, TAE226, PF-562271, PF-431396, VS-4718, PF-573228, BI853520, IN10018, and APG-2449;

alternatively, the FAK inhibitor is a compound represented by formula I, or optical isomers thereof, or tautomers thereof, or salts thereof, or prodrugs thereof, or hydrates thereof, or solvates thereof,

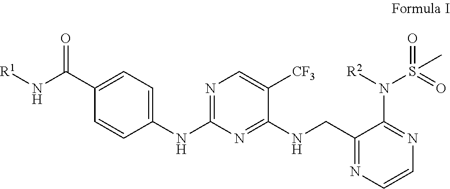

wherein, R¹ and R² are each independently selected from the group consisting of hydrogen, methyl, and trideuteromethyl;

alternatively, the FAK inhibitor is the following compounds, or optical isomers thereof, or tautomers thereof, or salts thereof, or prodrugs thereof, or hydrates thereof, or solvates thereof, and the following compounds are selected from the group consisting of:

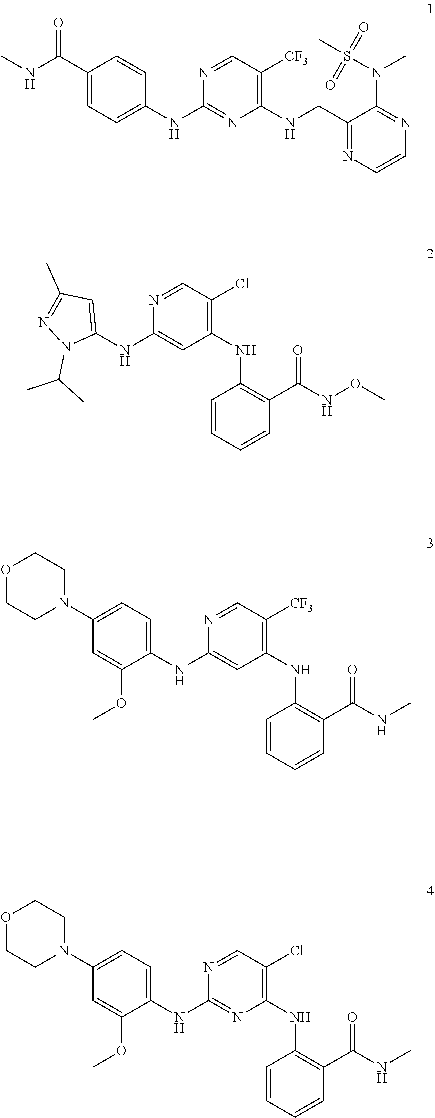

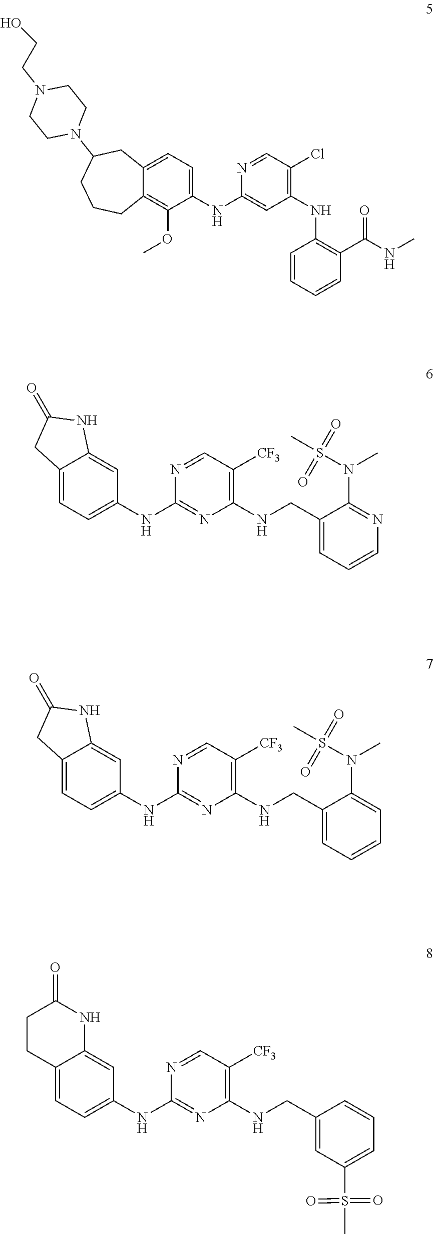 [SMILES: CNC(=O)c1ccccc1Nc1cc(Nc2ccc3c(c2OC)CCCC(N2CCN(CCO)CC2)C3)ncc1Cl ; CN(c1ncccc1CNc1nc(Nc2ccc3c(c2)NC(=O)C3)ncc1C(F)(F)F)S(C)(=O)=O ; CN(c1ccccc1CNc1nc(Nc2ccc3c(c2)NC(=O)C3)ncc1C(F)(F)F)S(C)(=O)=O ; CS(=O)(=O)c1cccc(CNc2nc(Nc3ccc4c(c3)NC(=O)CC4)ncc2C(F)(F)F)c1]

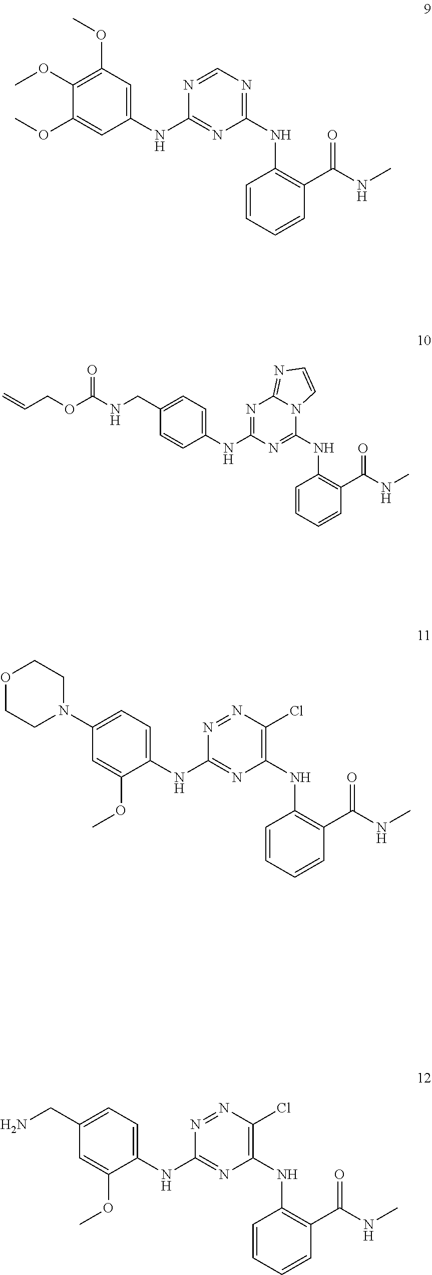

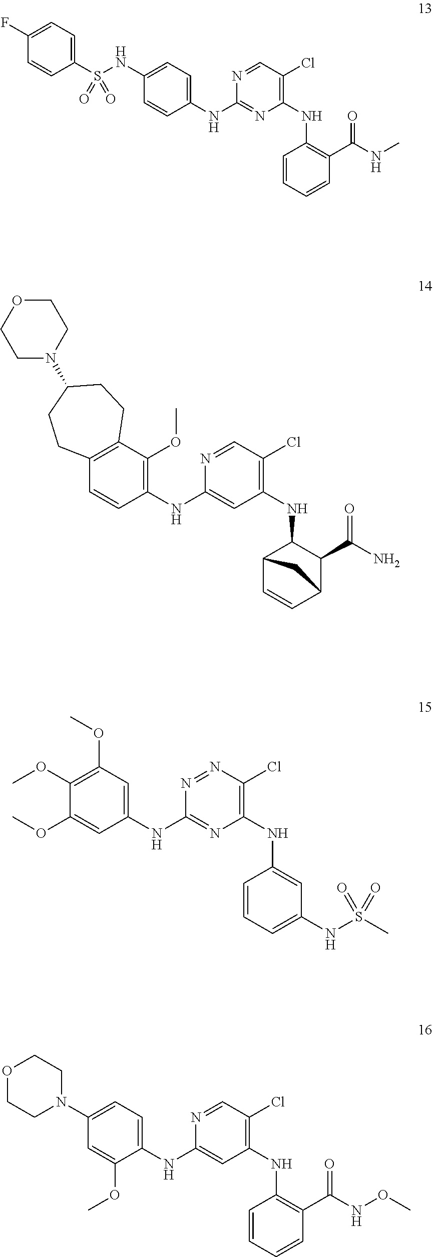

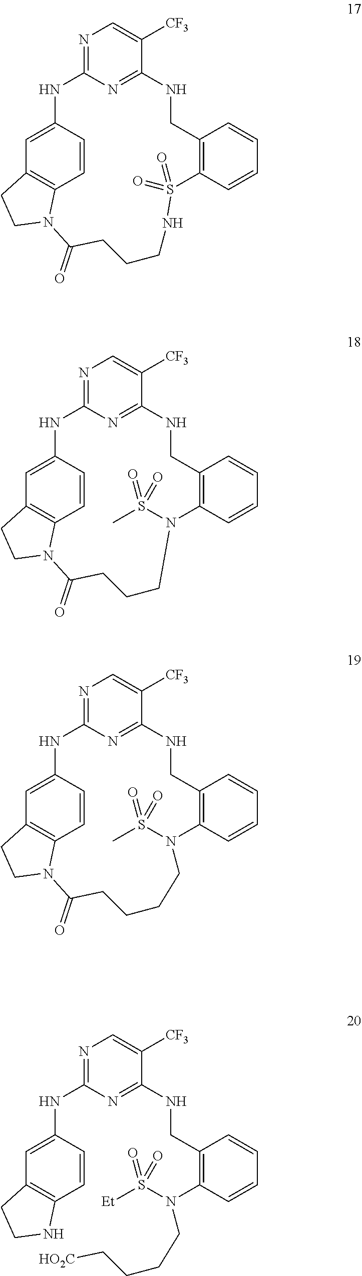 [SMILES: O=C1CCCNS(=O)(=O)c2ccccc2CNc2nc(ncc2C(F)(F)F)Nc2ccc3c(c2)CCN13 ; CS(=O)(=O)N1CCCC(=O)N2CCc3cc(ccc32)Nc2ncc(C(F)(F)F)c(n2)NCc2ccccc21 ; CS(=O)(=O)N1CCCCC(=O)N2CCc3cc(ccc32)Nc2ncc(C(F)(F)F)c(n2)NCc2ccccc21 ; CCS(=O)(=O)N(CCCCC(=O)O)c1ccccc1CNc1nc(Nc2ccc3c(c2)CCN3)ncc1C(F)(F)F]

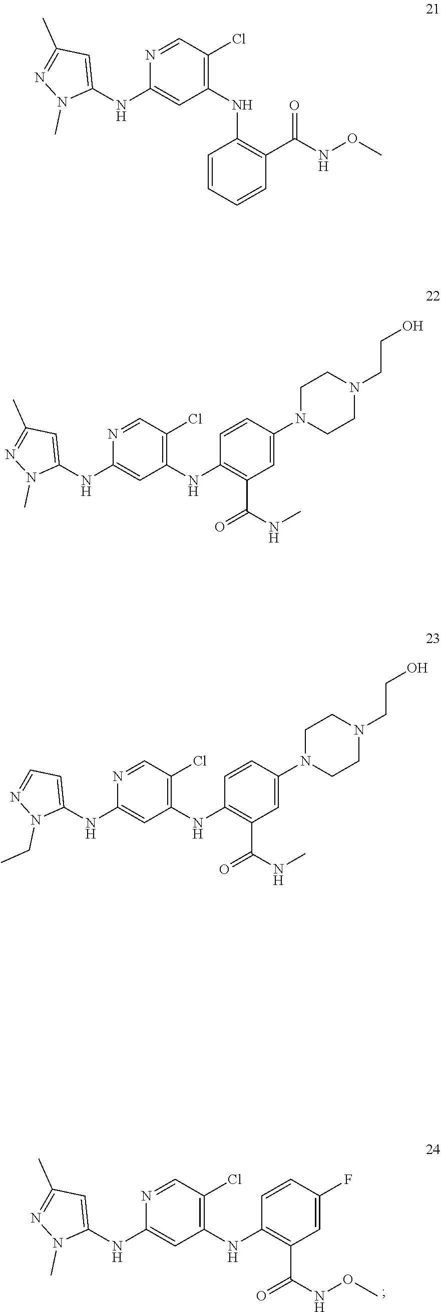

preferably, the FAK inhibitor is the following compounds, or optical isomers thereof, or tautomers thereof, or salts thereof, or prodrugs thereof, or hydrates thereof, or solvates thereof, and the following compounds include

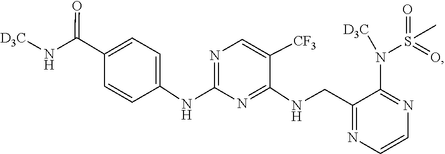 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]

### Defactinib or IN10018.

Further, the CD44 inhibitor is the following compounds, or optical isomers thereof, or tautomers thereof, or salts thereof, or prodrugs thereof, or hydrates thereof, or solvates thereof, or peptides, or antibodies, and the following compounds or peptides or antibodies include Angstrom6, PEP-1, AMC303, Anti-CD44 mAb H4C4, anti-CD44 mAb HI44a, anti-CD44 mAb IM7, anti-CD44 mAb KM201, RO5429083, RG7356, anti-CD44V6 mAb 2F10, anti-CD44V6 mAb U36, anti-CD44V6 mAb V6B3, anti-CD44V6 mAb VFF18, anti-CD44V6 mAb VFF4, anti-CD44V6 and mAb VFF7;

preferably, the CD44 inhibitor is the following compound, or optical isomers thereof, or tautomers thereof, or salts thereof, or prodrugs thereof, or hydrates thereof, or solvates thereof, and the following compound includes AMC303.

Further, the FAK inhibitor is compound

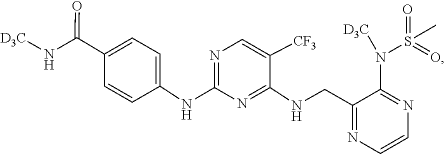 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]

or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-81:1;

alternatively, the FAK inhibitor is Defactinib, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-27:1;

alternatively, the FAK inhibitor is IN10018, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 1:1.

The present invention also provides the use of FAK inhibitors combined with CD44 inhibitors in the manufacturer of medicaments for treating cancer;

preferably, the weight ratio of FAK inhibitor and CD44 inhibitor is 1:10-10:1;

alternatively, the molar ratio of FAK inhibitor and CD44 inhibitor is 1:82-82:1;

more preferably, the molar ratio of FAK inhibitor and CD44 inhibitor is 0.4-81:1.

Further, the FAK inhibitors are the following compounds, or optical isomers thereof, or tautomers thereof, or salts thereof, or prodrugs thereof, or hydrates thereof, or solvates thereof, and the following compounds include: Defactinib, CEP-28122, CEP-37440, TAE226, PF-562271, PF-431396, VS-4718, PF-573228, BI853520, IN10018, and APG-2449;

alternatively, the FAK inhibitor is a compound represented by formula I, or optical isomers thereof, or tautomers thereof, or salts thereof, or prodrugs thereof, or hydrates thereof, or solvates thereof,

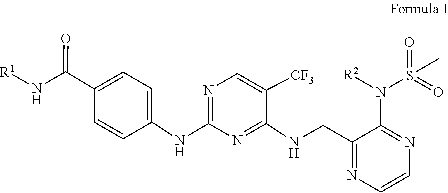

wherein, R¹ and R² are each independently selected from the group consisting of hydrogen, methyl, and trideuteromethyl;

alternatively, the FAK inhibitor is the following compounds, or optical isomers thereof, or tautomers thereof, or salts thereof, or prodrugs thereof, or hydrates thereof, or solvates thereof, and the following compounds are selected from the group consisting of:

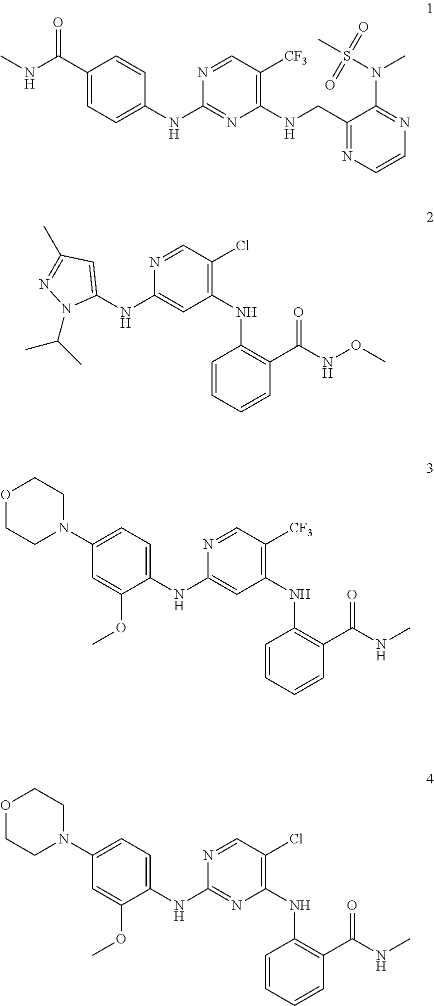

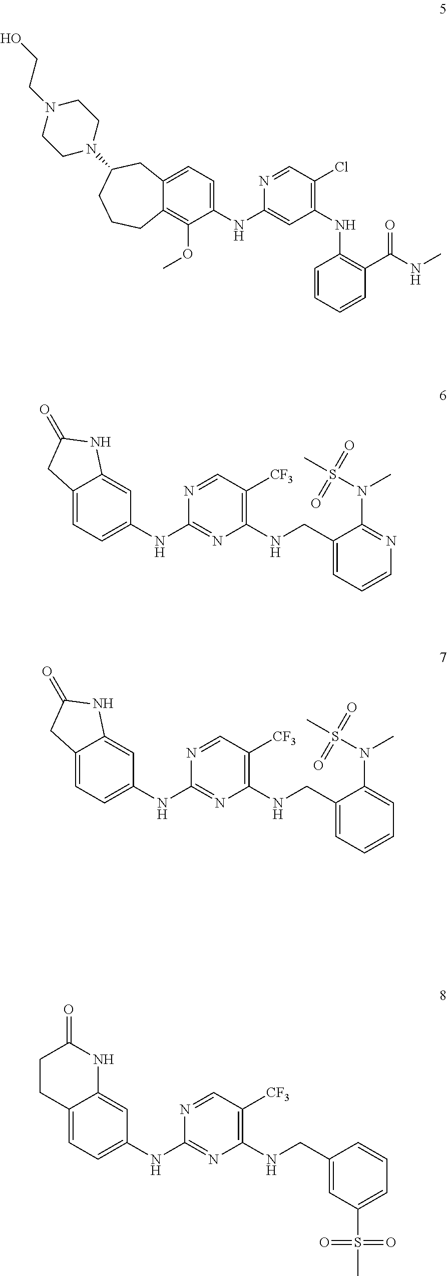 [SMILES: CNC(=O)c1ccccc1Nc1cc(Nc2ccc3c(c2OC)CCC[C@H](N2CCN(CCO)CC2)C3)ncc1Cl ; CN(c1ncccc1CNc1nc(Nc2ccc3c(c2)NC(=O)C3)ncc1C(F)(F)F)S(C)(=O)=O ; CN(c1ccccc1CNc1nc(Nc2ccc3c(c2)NC(=O)C3)ncc1C(F)(F)F)S(C)(=O)=O ; CS(=O)(=O)c1cccc(CNc2nc(Nc3ccc4c(c3)NC(=O)CC4)ncc2C(F)(F)F)c1]

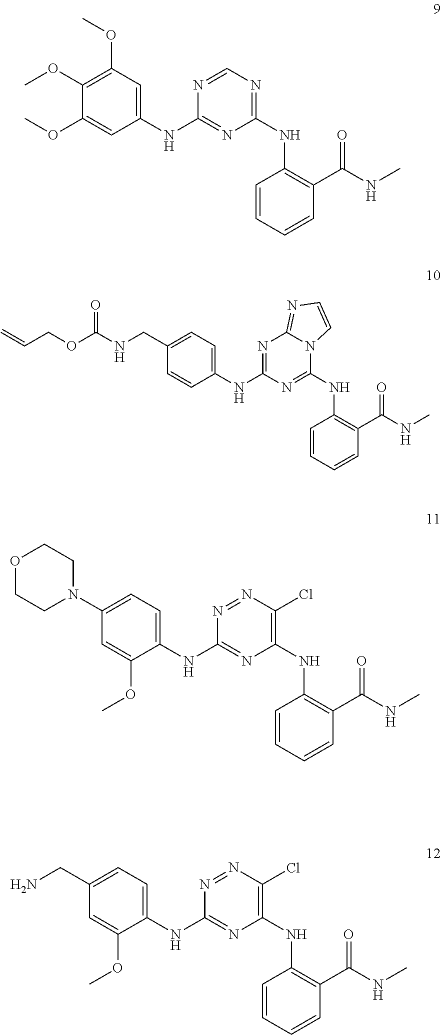

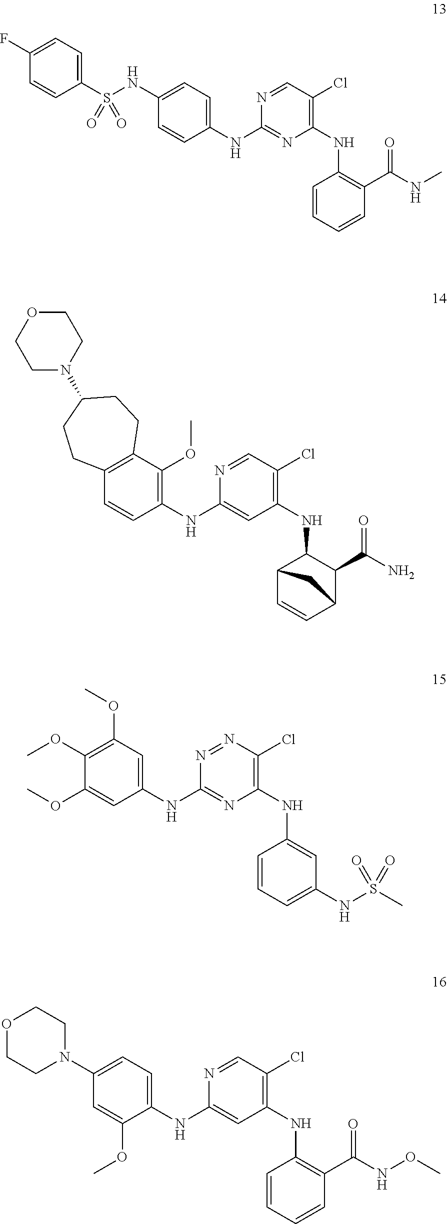

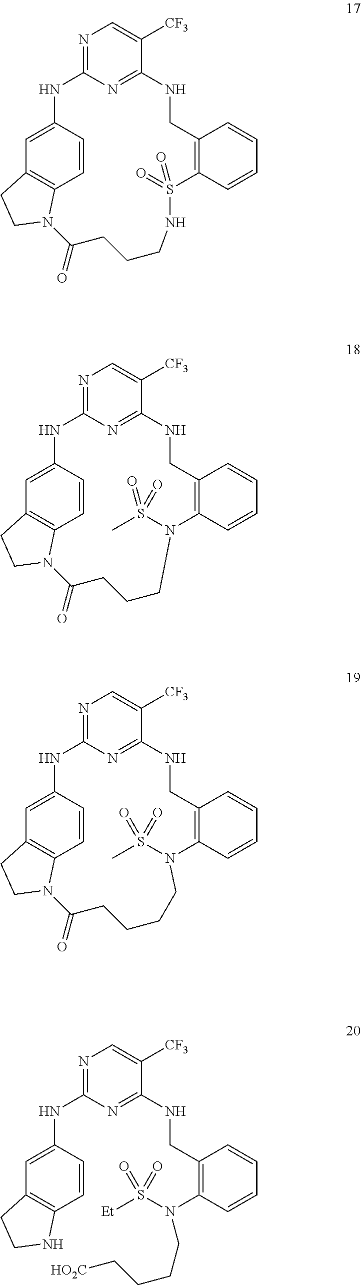 [SMILES: O=C1CCCNS(=O)(=O)c2ccccc2CNc2nc(ncc2C(F)(F)F)Nc2ccc3c(c2)CCN13 ; CS(=O)(=O)N1CCCC(=O)N2CCc3cc(ccc32)Nc2ncc(C(F)(F)F)c(n2)NCc2ccccc21 ; CS(=O)(=O)N1CCCCC(=O)N2CCc3cc(ccc32)Nc2ncc(C(F)(F)F)c(n2)NCc2ccccc21 ; CCS(=O)(=O)N(CCCCC(=O)O)c1ccccc1CNc1nc(Nc2ccc3c(c2)CCN3)ncc1C(F)(F)F]

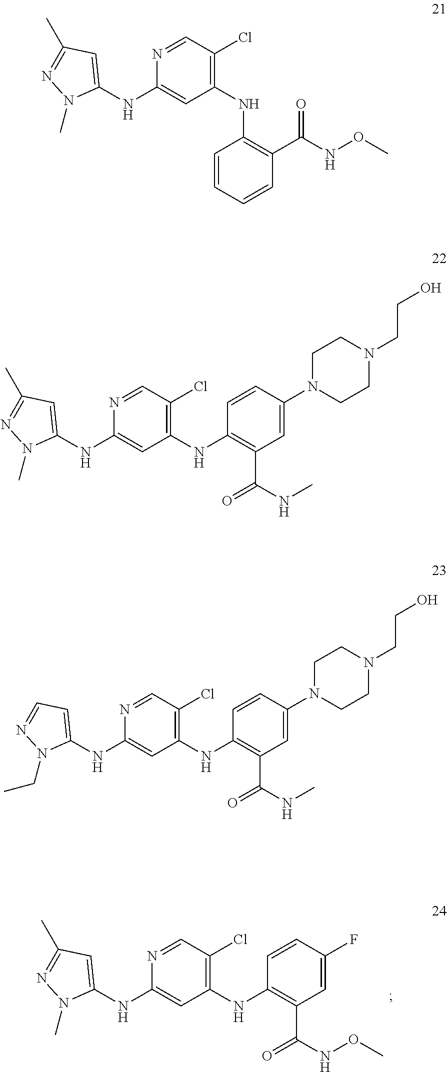

preferably, the FAK inhibitor is the following compounds, or optical isomers thereof, or tautomers thereof, or salts thereof, or prodrugs thereof, or hydrates thereof, or solvates thereof, and the following compounds include

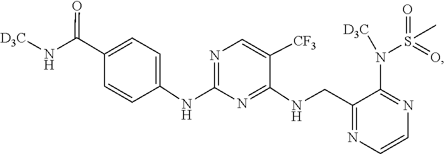 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]

### Defactinib or IN10018.

Further, the CD44 inhibitor is the following compounds, or optical isomers thereof, or tautomers thereof, or salts thereof, or prodrugs thereof, or hydrates thereof, or solvates thereof, or peptides, or antibodies, and the following compounds or peptides or antibodies include Angstrom6, PEP-1, AMC303, Anti-CD44 mAb H4C4, anti-CD44 mAb HI44a, anti-CD44 mAb IM7, anti-CD44 mAb KM201, RO5429083, RG7356, anti-CD44V6 mAb 2F10, anti-CD44V6 mAb U36, anti-CD44V6 mAb V6B3, anti-CD44V6 mAb VFF18, anti-CD44V6 mAb VFF4, anti-CD44V6, and mAb VFF7;

preferably, the CD44 inhibitor is the following compound, or optical isomers thereof, or tautomers thereof, or salts thereof, or prodrugs thereof, or hydrates thereof, or solvates thereof, and the following compound includes AMC303.

Further, the FAK inhibitor is compound

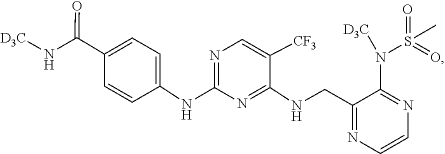 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]

or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-81:1;

alternatively, the FAK inhibitor is Defactinib, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-27:1;

alternatively, the FAK inhibitor is IN10018, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 1:1.

Further, the cancer is solid tumor, mesothelioma, melanoma, prostate cancer, breast cancer, glioblastoma, and brain cancer;

the solid tumors include mesothelioma, pancreatic cancer, soft tissue tumor, metastatic tumor, non-solid cancer, sarcoma, adenocarcinoma, lung cancer, breast cancer, lymphoma, gastrointestinal cancer, urogenital system cancer, prostate cancer, ovarian cancer; the gastrointestinal cancer includes colon cancer, and the urogenital system cancer includes renal, urothelial or testicular tumors; the ovarian cancer includes advanced ovarian cancer;

the mesothelioma includes neurofibroma, renal cancer, lung cancer, small cell lung cancer, non-small cell lung cancer, KRAS-mutant non-small cell lung cancer, liver cancer, thyroid cancer, breast cancer, nervous system tumor, neurilemmoma, meningioma, neuroma, adenoid cystic carcinoma, ependymoma, ependymal tumor, malignant pleural tumor, malignant pleural mesothelioma, triplet tumor, negative breast cancer, non-blood malignant tumor, melanoma, colorectal cancer, leukemia, adenocarcinoma, and solid tumors;

the melanoma includes locally advanced melanoma, locally mutant N-Ras driven melanoma, and metastatic malignant skin melanoma; the colorectal cancer includes metastatic colorectal cancer; the leukemia includes acute myelogenous leukemia; the adenocarcinoma includes adenocarcinoma; the solid tumors include locally advanced solid tumors, metastatic solid tumors, and hepatocellular carcinoma;

the prostate cancer includes castration-resistant prostate cancer and metastatic castration resistant prostate cancer;

the brain cancer includes neuroepithelial tissue tumors, cerebral gliomas, astrocytomas, oligodendrogliomas, ependymal and choroidal plexus tumors, pineal tumors, neurocellular tumors, gangliocytoma, neuroblastoma, poorly differentiated tumors, embryonal tumors, glioblastoma multiform, medulloblastoma, schwannoma, meningioma, malignant lymphoma, cerebrovascular tumors, vascular malformations capillary telangiectases, pituitary tumors, and metastatic tumors.

The drug combination of CD44 inhibitors and FAK inhibitors according to the present invention can play a synergistic effect, significantly improve the inhibitory effect on tumors, reduce toxic side effects, and overcome tumor drug resistance, thereby providing a good choice for clinical treatment of cancer.

Obviously, based on the above content of the present invention, according to the common technical knowledge and the general means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Histogram of survival rate for compound 25 combined with AMC303 against A549 cells.

FIG. 2. Histogram of survival rate for Defactinib combined with AMC303 against A549 cells.

FIG. 3. Images of wound healing assay for compound 25, Defactinib or IN10018 combined with AMC303 against A549 cells.

FIG. 4. Statistical chart of wound healing assay for compound 25, Defactinib or IN10018 combined with AMC303 against A549 cells.

FIG. 5. Histogram of survival rate for compound 25 combined with AMC303 against SW620 cells.

FIG. 6. Histogram of survival rate for Defactinib combined with AMC303 against SW620 cells.

### EXAMPLES

The starting materials and equipment used in the specific examples of the present invention are known products obtained by purchasing those commercially available.

In the examples of the present invention, the structure of compound 25 is

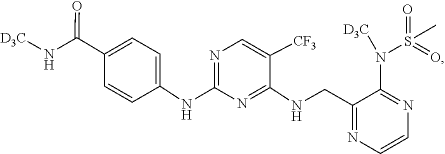 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]

which was prepared according to the method disclosed in the patent application number 201910373081.2. Defactinib is a commercially available FAK inhibitor. AMC303 is a commercially available CD44v6 inhibitor developed by amcure GmbH. IN10018 is a commercially available FAK inhibitor.

### Example 1: The Inhibitory Effect of the Pharmaceutical Composition According to the Present Invention on the Proliferation of Cancer Cells

### 1. Experimental Methods

Non-small cell lung cancer A549 cells in the logarithmic growth phase were seeded in a 96-well plate at a concentration of 1.0×10³ cells/well. The plate was placed in an incubator and cultured at 37° C. under 5% CO₂ and saturated humidity for 24 h. After 24 hours of incubation, the drugs FAK inhibitor (compound 25 or Defactinib) and CD44 inhibitor (AMC303) were added successively to the 96-well plate. RPMI 1640 cell culture medium containing 10% fetal bovine serum was added to the negative control wells in a volume same as that of the drug.

The concentration of compound 25 was: 0 μM, 0.123 μM, 0.370 μM, 1.111 μM, 3.333 μM, and 10 μM

The concentration of Defactinib was: 0 μM, 0.123 μM, 0.370 μM, 1.111 μM, 3.333 μM, and 10 μM

The concentration of AMC303 was: 0 μM, 0.002 μM, 0.005 μM, 0.014 μM, 0.041 μM, 0.123 μM, 0.370 μM, 1.111 μM, 3.333 μM, and 10 μM.

After co-incubation with drugs for 6 days, 10 μL of CCK-8 was added to each well, followed by cultivating for additional 2.5 h. The absorbance value (OD) of each well was measured using an enzyme-linked immunosorbent assay (ELISA) at a wavelength of 450 nm. The inhibition rate of cell growth was calculated according to the formula, that is, Inhibition rate (%)=(1−OD value of test well/OD value of control well)×100%. Then, the effect was determined based on the drug combination index q=Ea+b/(Ea+Eb−Ea*Eb). The judgment criteria for q value is shown in Table 1:

TABLE 1
Judgment criteria for q value.

| q | Description |
|---|---|
| >1 | Synergistic effect |
| <1 | Antagonistic effect |

### 2. Experimental Results

### (1) Combination of Compound 25 and AMC303 (Compound 25 & AMC303)

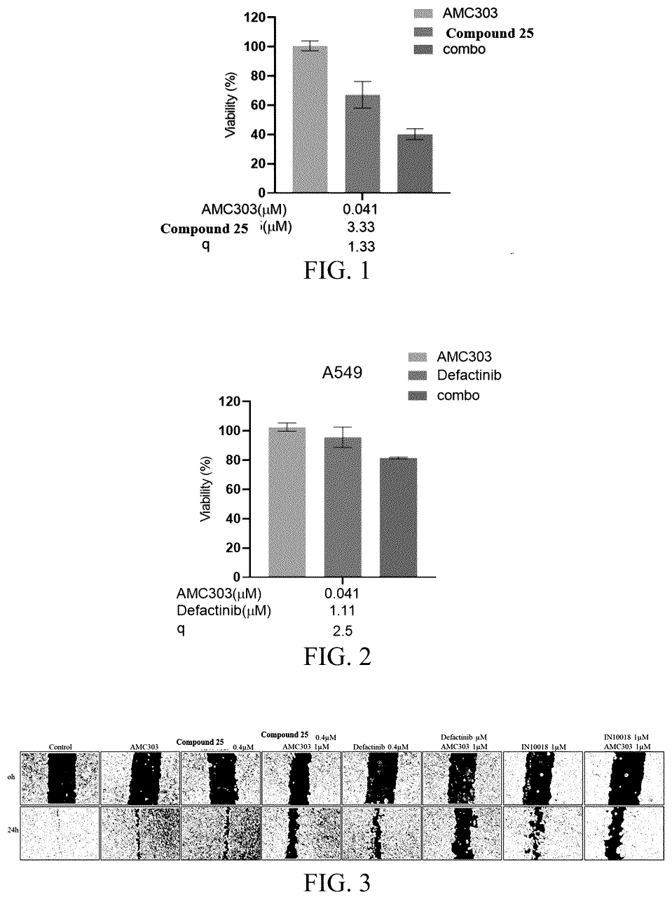

The results for the combination of compound 25 and AMC303 are shown in FIG. 1. In FIG. 1, when the molar ratio of compound 25 and AMC303 was 81:1, the combination of compound 25 and AMC303 had a synergistic effect on inhibiting the growth of A549 cells, and thereby could be used for synergistic treatment of non-small cell lung cancer.

### (2) Combination of Defactinib and AMC303 (Defactinib&AMC303)

The results for the combination of Defactinib and AMC303 are shown in FIG. 2. As shown in FIG. 2, when the molar ratio of Defactinib and AMC303 was 27:1, the drug combination of Defactinib and AMC303 had a synergistic effect on inhibiting the growth of A549 cells, and thereby could be used for synergistically treating non-small cell lung cancer.

The above example provided a drug combination for the treatment of non-small cell lung cancer. The experimental results indicated that the combination of FAK inhibitor (compound 25 or Defactinib) and CD44 inhibitor AMC303 could significantly inhibit the growth and proliferation of non-small cell lung cancer cells, and the anticancer effect of the combination was significantly better than that of FAK inhibitor (compound 25 or Defactinib) and CD44 inhibitor AMC303 used alone, suggesting a synergistic effect. The drug combination of the present invention has good clinical application prospects in the treatment of non-small cell lung cancer.

### Example 2: The Effect of the Pharmaceutical Composition According to the Present Invention on Inhibiting the Migration of Cancer cells

### 1. Experimental Method

Non-small cell lung cancer A549 cells in logarithmic growth phase were collected, and then seeded into a 12-well plate at a concentration of 1.0×10⁶ cells/well. The plate was placed in an incubator at 37° C. under 5% CO₂ and saturated humidity, and then cultured for 24 hours. The cells in the 12-well plate were assigned with 3 scratches per well. After scratching, the media were discarded, and each well was further rinsed with PBS to remove floating cells. FAK inhibitors (compound 25, Defactinib, or IN10018) and CD44 inhibitors (AMC303) were successively added to the 12-well plate. The drugs were dissolved in serum-free RPMI 1640 cell culture medium containing 20 ng/ml HGF. Serum-free RPMI 1640 cell culture medium (containing 20 ng/ml HGF) was also added to the negative control well in the same volume as that of the drug solution.

Concentration of compound 25: 0.4 μM;

Concentration of Defactinib: 0.4 μM;

Concentration of IN10018: 1 μM;

Concentration of AMC303: 1 μM.

After adding 4 drugs separately (compound 25, Defactinib, IN10018, AMC303), or simultaneously adding compound 25 and AMC303 (compound 25&AMC303, at a concentration of 0.4 μM for compound 25 and 1 μM for AMC303), or simultaneously adding Defactinib and AMC303 (Defactinib&AMC303, at a concentration of 0.4 μM for Defactinib and 1 μM for AMC303), or simultaneously adding IN10018 and AMC303 (IN10018&AMC303, at a concentration of 1 μM for IN10018 and 1 μM for AMC303), the plate was incubated for 24 hours, and then the culture medium was discarded. The plate was rinsed with PBS to remove floating cells. The migration of cells was observed under a microscope, and the image was produced by the action of eyepiece 10× and objective 10× (images were taken at 0 h and 24 h, respectively). Image processing was performed using Image J, to analyze the migration rate of cells. The formula for calculating the migration rate:

$$Migration rate ( \% ) = [ Scratch area ( 0 h ) - Scratch area ( 48 h ) ] / Scratch area ( 0 h ) \times 100 \%$$

Based on the migration rate, a histogram was created by Prism Statistics, and then T-test was carried out. p<0.05 means a significant difference.

### 2. Experimental Results

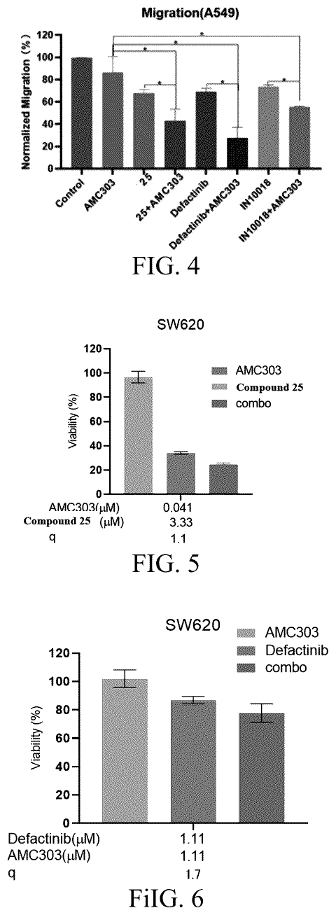

The results of cell migration are shown in FIGS. 3-4 and Table 2.

TABLE 2
The statistics of significant differences in cell migration
results among groups.

| Pharmaceutical compositions | P values |
|---|---|
| Compound 25&AMC303 vs Compound 25 | 0.046 |
| Compound 25&AMC303 vs AMC303 | 0.036 |
| Defactinib&AMC303 vs Defactinib | 0.028 |
| Defactinib&AMC303 vs AMC303 | 0.016 |
| IN10018&AMC303 vs IN10018 | 0.045 |
| IN10018&AMC303 vs AMC303 | 0.014 |

As shown in FIGS. 3-4 and Table 2, the combination of FAK inhibitors (compound 25, Defactinib, or IN10018) and CD44 inhibitor AMC303 could significantly inhibit the migration of non-small cell lung cancer cells, and the anticancer effect of the combination was significantly better than that of FAK inhibitors (compound 25, Defactinib, or IN10018) and CD44 inhibitor AMC303 alone, indicating a synergistic effect. The drug combination of the present invention had good clinical application prospects in the treatment of non-small cell lung cancer.

### Example 3: The Inhibitory Effect of the Pharmaceutical Composition According to the Present Invention on the Proliferation of Cancer Cells

### 1. Experimental Methods

Colorectal cancer SW620 cells in logarithmic growth phase were seeded in a 96-well plate at a concentration of 1.0×10³ cells/well. The plate was cultured in an incubator at 37° C.under 5% CO₂ and saturated humidity for 24 h. After 24 hours of incubation, the drugs FAK inhibitor (compound 25 or Defactinib) and CD44 inhibitor (AMC303) were added successively to the 96-well plate. RPMI 1640 cell culture medium containing 10% fetal bovine serum was added to the negative control wells in a volume same as that of the drug.

The concentration of compound 25 was: 0 μM, 0.123 μM, 0.370 μM, 1.111 μM, 3.333 μM, and 10 μM

The concentration of Defactinib was: 0 μM, 0.123 μM, 0.370 μM, 1.111 μM, 3.333 μM, and 10 μM

The concentration of AMC303 was: 0 μM, 0.002 μM, 0.005 μM, 0.014 μM, 0.041 μM, 0.123 μM, 0.370 μM, 1.111 μM, 3.333 μM, and 10 μM.

After co-incubation with drugs for 6 days, 10 μL of CCK-8 was added to each well, followed by culturing for additional 2.5 h. The absorbance value (OD) of each well was measured with ELISA at a wavelength of 450 nm. The inhibition rate of cell growth was calculated according to the formula:

$$Inhibition rate ( \% ) = ( 1 ‐ OD value of test well / OD value of control well ) \times 100 \% .$$

Then, the effect was determined based on the drug combination index q=Ea+b/(Ea+Eb−Ea*Eb). The judgment criteria for q value is shown in Table 3:

TABLE 3
Judgment criteria for q value.

| q | Description |
|---|---|
| >1 | Synergistic effect |
| <1 | Antagonistic effect |

### 2. Experimental Results

### (1) Combination of Compound 25 and AMC303 (Compound 25&AMC303)

The results for the combination of compound 25 and AMC303 are shown in FIG. 5. Based on FIG. 1, when the molar ratio of compound 25 and AMC303 was 81:1, the combination of compound 25 and AMC303 had a synergistic effect on inhibiting the growth of SW620 cells, and thus could be used for synergistically treating colorectal cancer.

### (2) Combination of Defactinib and AMC303 (Defactinib&AMC303)

The results for the combination of Defactinib and AMC303 are shown in FIG. 6. As shown in FIG. 6, when the molar ratio of Defactinib and AMC303 was 1:1, the combination of Defactinib and AMC303 had a synergistic effect on inhibiting the growth of SW620 cells, and so could be used for synergistically treating colorectal cancer.

The above example provided a drug combination for the treatment of colorectal cancer. The experimental results indicated that the combination of FAK inhibitor (compound 25 or Defactinib) and CD44 inhibitor AMC303 could significantly inhibit the growth and proliferation of colorectal cells, and the anticancer effect of the combination was significantly better than that of FAK inhibitor (compound 25 or Defactinib) and CD44 inhibitor AMC303 alone, suggesting a synergistic effect. The drug combination of the present invention had good clinical application prospects in the treatment of colorectal cancer.

In summary, the inventors had found that the combination of CD44 inhibitor and FAK inhibitor was demonstrated to have a synergistic effect, significantly improved the inhibitory effect on tumors, and overcame tumor drug resistance, thereby providing a good choice for clinical treatment of cancer.

1. A pharmaceutical composition for treating cancers, characterized in that it is composed of FAK inhibitors and CD44 inhibitors.

2. The pharmaceutical composition according to claim 1, characterized in that the weight ratio of FAK inhibitor and CD44 inhibitor is 1:10-10:1.

3. The pharmaceutical composition according to claim 1, characterized in that the FAK inhibitors are the following compounds, or optical isomers thereof, or tautomers thereof, or salts thereof, or prodrugs thereof, or hydrates thereof, or solvates thereof, and the following compounds include: Defactinib, CEP-28122, CEP-37440, TAE226, PF-562271, PF-431396, VS-4718, PF-573228, BI853520, IN10018, and APG-2449;
alternatively, the FAK inhibitor is a compound represented by formula I, or optical isomers thereof, or tautomers thereof, or salts thereof, or prodrugs thereof, or hydrates thereof, or solvates thereof, 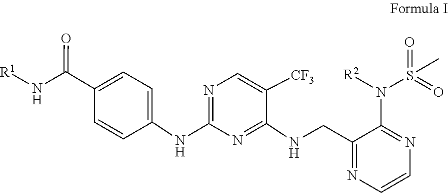
wherein, R¹ and R² are each independently selected from the group consisting of hydrogen, methyl, and trideuteromethyl;
alternatively, the FAK inhibitor is the following compounds, or optical isomers thereof, or tautomers thereof, or salts thereof, or prodrugs thereof, or hydrates thereof, or solvates thereof, and the following compounds are selected from the group consisting of: 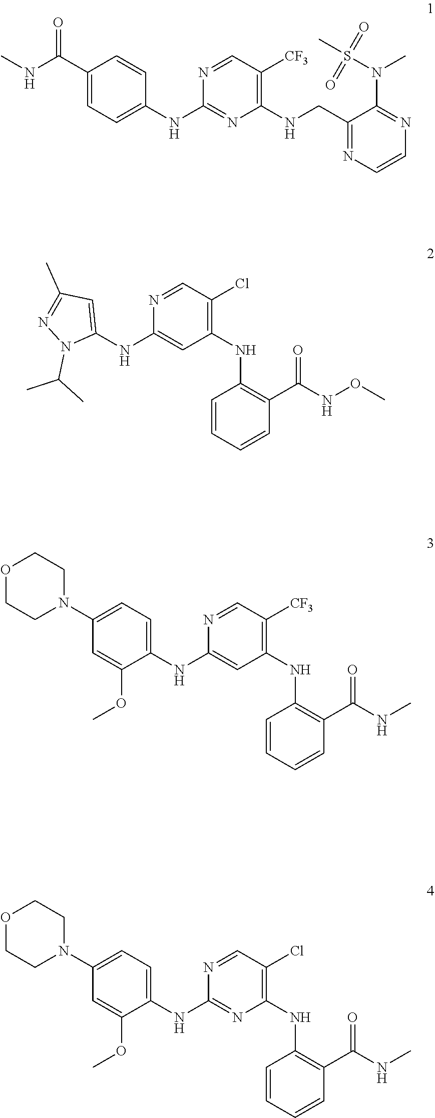 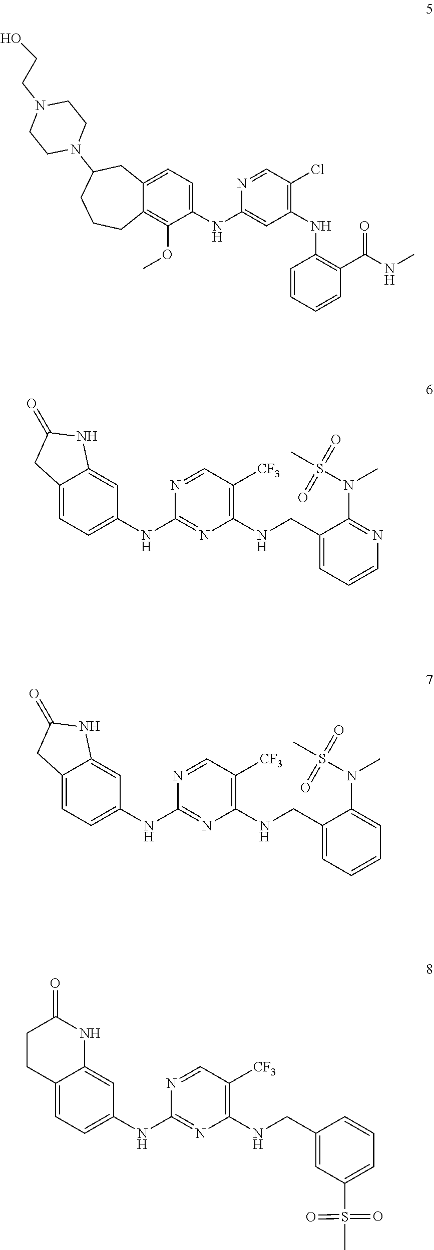 [SMILES: CNC(=O)c1ccccc1Nc1cc(Nc2ccc3c(c2OC)CCCC(N2CCN(CCO)CC2)C3)ncc1Cl ; CN(c1ncccc1CNc1nc(Nc2ccc3c(c2)NC(=O)C3)ncc1C(F)(F)F)S(C)(=O)=O ; CN(c1ccccc1CNc1nc(Nc2ccc3c(c2)NC(=O)C3)ncc1C(F)(F)F)S(C)(=O)=O ; CS(=O)(=O)c1cccc(CNc2nc(Nc3ccc4c(c3)NC(=O)CC4)ncc2C(F)(F)F)c1] 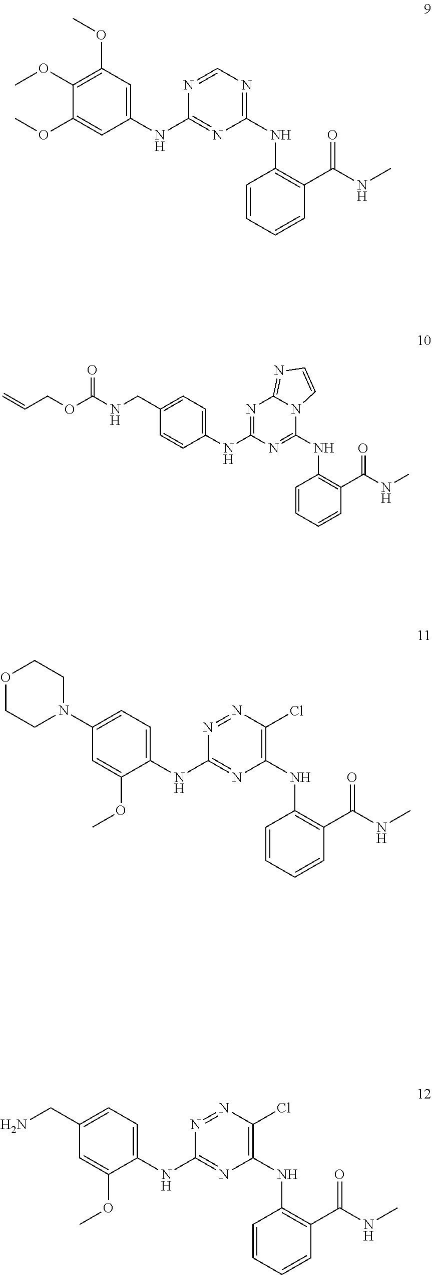 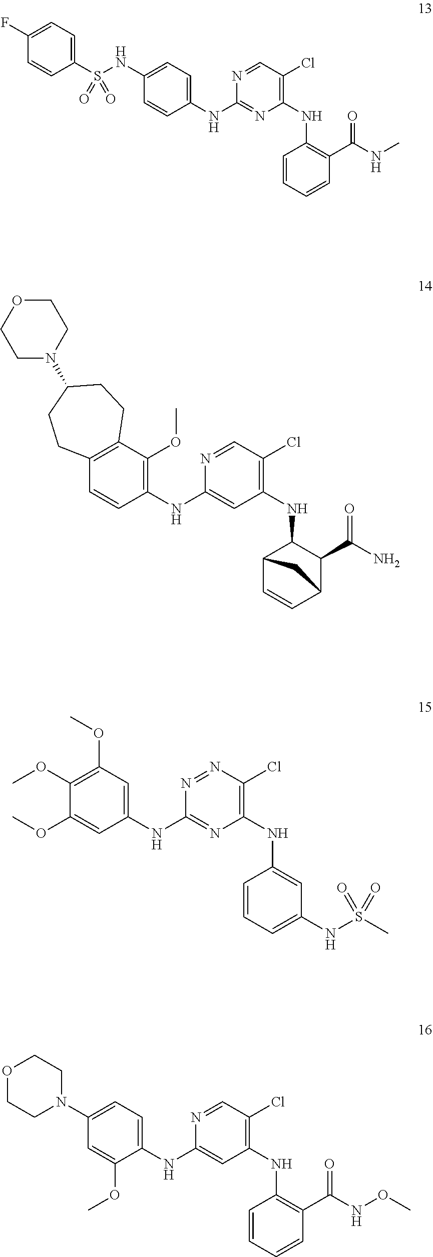 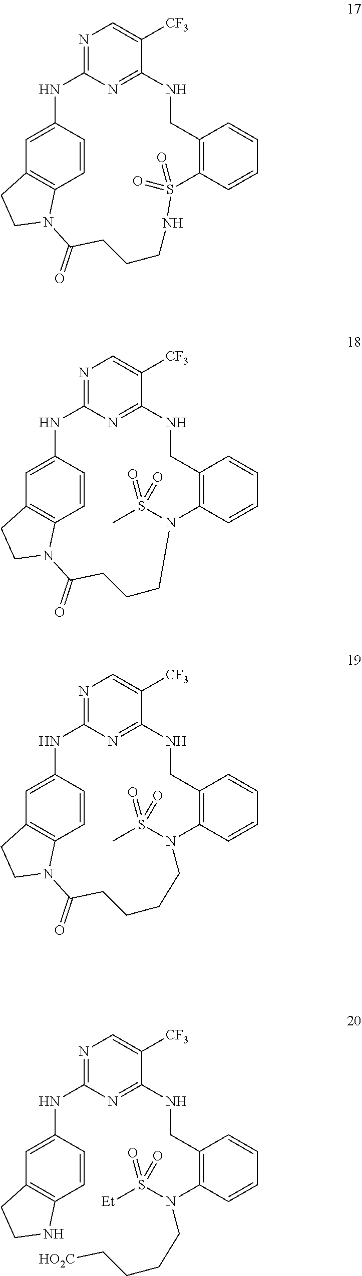 [SMILES: O=C1CCCNS(=O)(=O)c2ccccc2CNc2nc(ncc2C(F)(F)F)Nc2ccc3c(c2)CCN13 ; CS(=O)(=O)N1CCCC(=O)N2CCc3cc(ccc32)Nc2ncc(C(F)(F)F)c(n2)NCc2ccccc21 ; CS(=O)(=O)N1CCCCC(=O)N2CCc3cc(ccc32)Nc2ncc(C(F)(F)F)c(n2)NCc2ccccc21 ; CCS(=O)(=O)N(CCCCC(=O)O)c1ccccc1CNc1nc(Nc2ccc3c(c2)CCN3)ncc1C(F)(F)F] 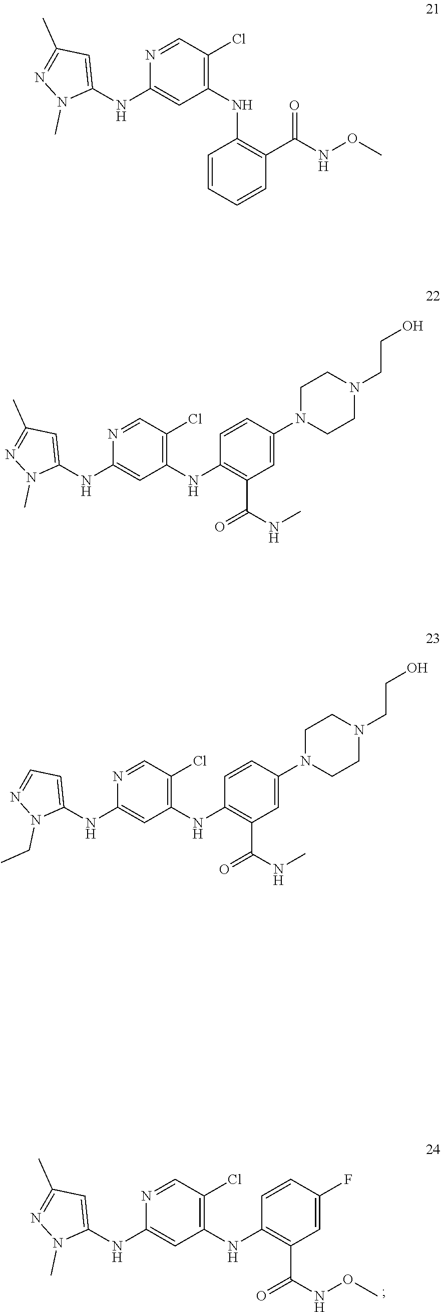
preferably, the FAK inhibitor is the following compounds, or optical isomers thereof, or tautomers thereof, or salts thereof, or prodrugs thereof, or hydrates thereof, or solvates thereof, and the following compounds include 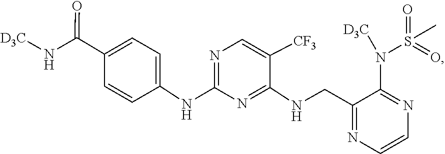 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]
Defactinib or IN10018.

4. The pharmaceutical composition according to claim 1, characterized in that the CD44 inhibitor is the following compounds, or optical isomers thereof, or tautomers thereof, or salts thereof, or prodrugs thereof, or hydrates thereof, or solvates thereof, or peptides, or antibodies, and the following compounds or peptides or antibodies include Angstrom6, PEP-1, AMC303, Anti-CD44 mAb H4C4, anti-CD44 mAb HI44a, anti-CD44 mAb IM7, anti-CD44 mAb KM201, RO5429083, RG7356, anti-CD44V6 mAb 2F10, anti-CD44V6 mAb U36, anti-CD44V6 mAb V6B3, anti-CD44V6 mAb VFF18, anti-CD44V6 mAb VFF4, anti-CD44V6 and mAb VFF7;
preferably, the CD44 inhibitor is the following compound, or optical isomers thereof, or tautomers thereof, or salts thereof, or prodrugs thereof, or hydrates thereof, or solvates thereof, and the following compound includes AMC303.

5. The pharmaceutical composition according to claim 1, characterized in that the molar ratio of FAK inhibitor and CD44 inhibitor is 1:82-82:1;
preferably, the molar ratio of FAK inhibitor and CD44 inhibitor is 0.4-81:1.

6. The pharmaceutical composition according to claim 1, characterized in that the FAK inhibitor is compound 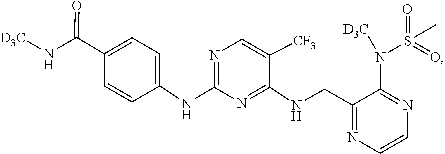 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]
or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-81:1;
alternatively, the FAK inhibitor is Defactinib, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-27:1;
alternatively, the FAK inhibitor is IN10018, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 1:1.

7. A preparation method for a pharmaceutical composition according to claim 1, characterized in that it comprises the following steps: FAK inhibitors and CD44 inhibitors were weighed according to the pre-determined weight ratio, and then well mixed.

8. The pharmaceutical composition according to claim 1 for use in the manufacturer of medicaments for treating cancers;
preferably, the cancer is solid tumor, mesothelioma, melanoma, prostate cancer, breast cancer, glioblastoma, and brain cancer;
the solid tumors include mesothelioma, pancreatic cancer, soft tissue tumor, metastatic tumor, non-solid cancer, sarcoma, adenocarcinoma, lung cancer, breast cancer, lymphoma, gastrointestinal cancer, urogenital system cancer, prostate cancer, ovarian cancer; the gastrointestinal cancer includes colon cancer, and the urogenital system cancer includes renal, urothelial, or testicular tumors; the ovarian cancer includes advanced ovarian cancer;
the mesothelioma includes neurofibroma, renal cancer, lung cancer, small cell lung cancer, non-small cell lung cancer, KRAS-mutant non-small cell lung cancer, liver cancer, thyroid cancer, breast cancer, nervous system tumor, neurilemmoma, meningioma, neuroma, adenoid cystic carcinoma, ependymoma, ependymal tumor, malignant pleural tumor, malignant pleural mesothelioma, triplet tumor, negative breast cancer, non-blood malignant tumor, melanoma, colorectal cancer, leukemia, adenocarcinoma, and solid tumors;
the melanoma includes locally advanced melanoma, locally mutant N-Ras driven melanoma, and metastatic malignant skin melanoma; the colorectal cancer includes metastatic colorectal cancer; the leukemia includes acute myelogenous leukemia; the adenocarcinoma includes adenocarcinoma; the solid tumors include locally advanced solid tumors, metastatic solid tumors, and hepatocellular carcinoma;
the prostate cancer includes castration-resistant prostate cancer and metastatic castration resistant prostate cancer;
the brain cancer includes neuroepithelial tissue tumors, cerebral gliomas, astrocytomas, oligodendrogliomas, ependymal and choroidal plexus tumors, pineal tumors, neurocellular tumors, gangliocytoma, neuroblastoma, poorly differentiated tumors, embryonal tumors, glioblastoma multiform, medulloblastoma, schwannoma, meningioma, malignant lymphoma, cerebrovascular tumors, vascular malformations, capillary telangiectases, pituitary tumors, and metastatic tumors.

9. A pharmaceutical preparation for treating cancers, characterized in that it is made from the pharmaceutical compositions according to claim 1, as active ingredients, in combination with pharmaceutically acceptable excipients or auxiliary ingredients.

10. A drug combination for treating cancers, characterized in that it comprises FAK inhibitors and CD44 inhibitors in the same or different specifications administered simultaneously or separately, as well as pharmaceutically acceptable carriers;
preferably, the weight ratio of FAK inhibitor and CD44 inhibitor is 1:10-10:1;
alternatively, the molar ratio of FAK inhibitor and CD44 inhibitor is 1:82-82:1;
more preferably, the molar ratio of FAK inhibitor and CD44 inhibitor is 0.4-81:1.

11. The combination drug according to claim 10, characterized in that the FAK inhibitors are the following compounds, or optical isomers thereof, or tautomers thereof, or salts thereof, or prodrugs thereof, or hydrates thereof, or solvates thereof, and the following compounds include: Defactinib, CEP-28122, CEP-37440, TAE226, PF-562271, PF-431396, VS-4718, PF-573228, BI853520, IN10018, and APG-2449;
alternatively, the FAK inhibitor is a compound represented by formula I, or optical isomers thereof, or tautomers thereof, or salts thereof, or prodrugs thereof, or hydrates thereof, or solvates thereof, 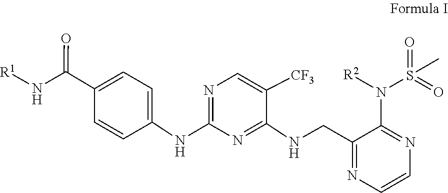
wherein, R¹ and R² are each independently selected from the group consisting of hydrogen, methyl, and trideuteromethyl;
alternatively, the FAK inhibitor is the following compounds, or optical isomers thereof, or tautomers thereof, or salts thereof, or prodrugs thereof, or hydrates thereof, or solvates thereof, and the following compounds are selected from the group consisting of: 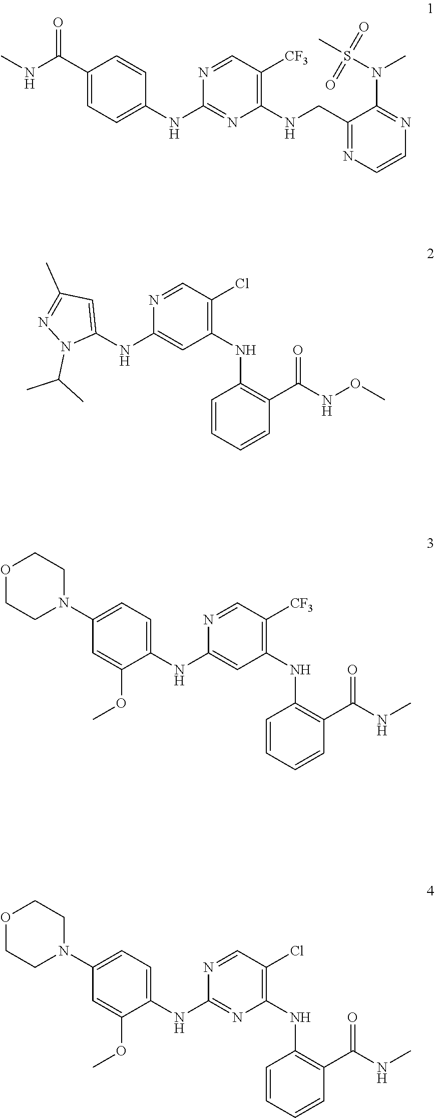 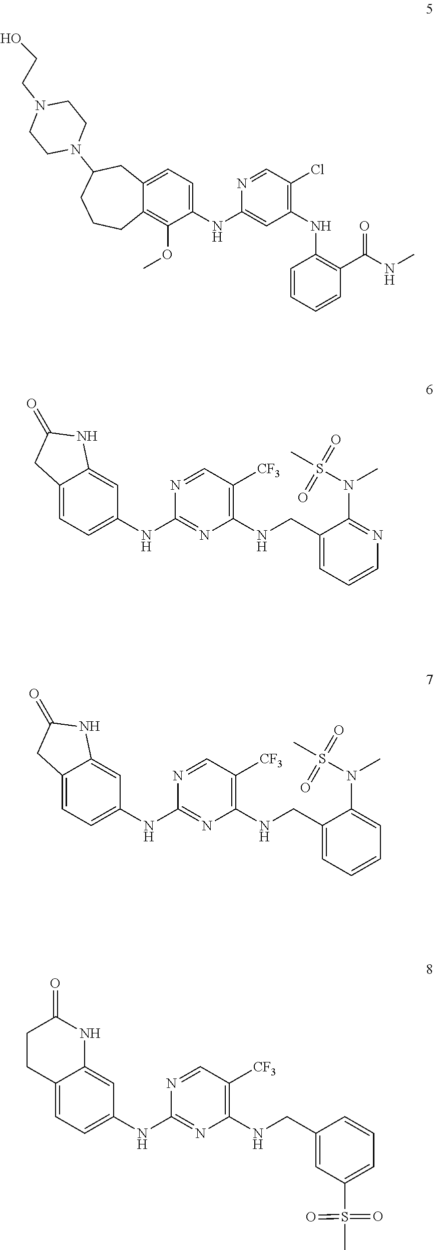 [SMILES: CNC(=O)c1ccccc1Nc1cc(Nc2ccc3c(c2OC)CCCC(N2CCN(CCO)CC2)C3)ncc1Cl ; CN(c1ncccc1CNc1nc(Nc2ccc3c(c2)NC(=O)C3)ncc1C(F)(F)F)S(C)(=O)=O ; CN(c1ccccc1CNc1nc(Nc2ccc3c(c2)NC(=O)C3)ncc1C(F)(F)F)S(C)(=O)=O ; CS(=O)(=O)c1cccc(CNc2nc(Nc3ccc4c(c3)NC(=O)CC4)ncc2C(F)(F)F)c1] 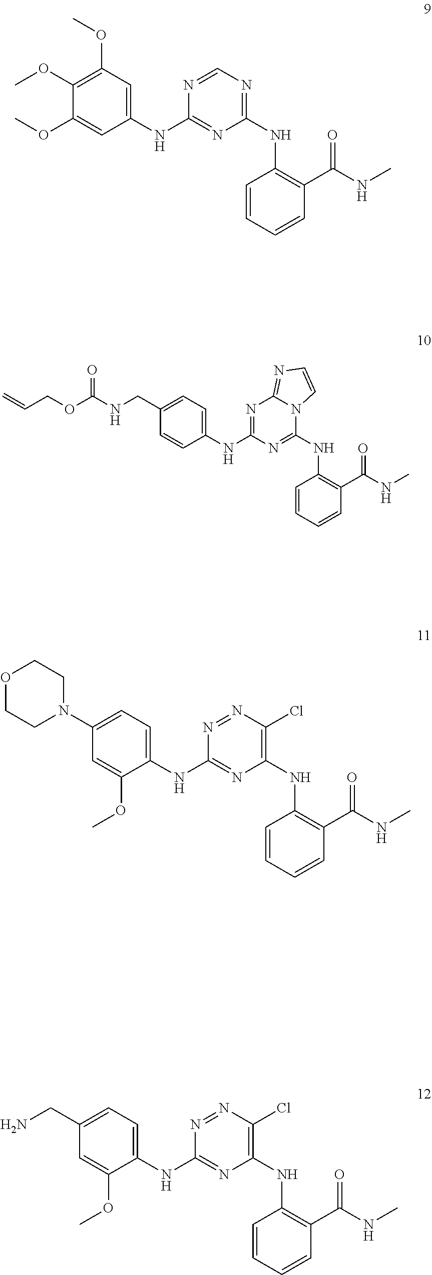 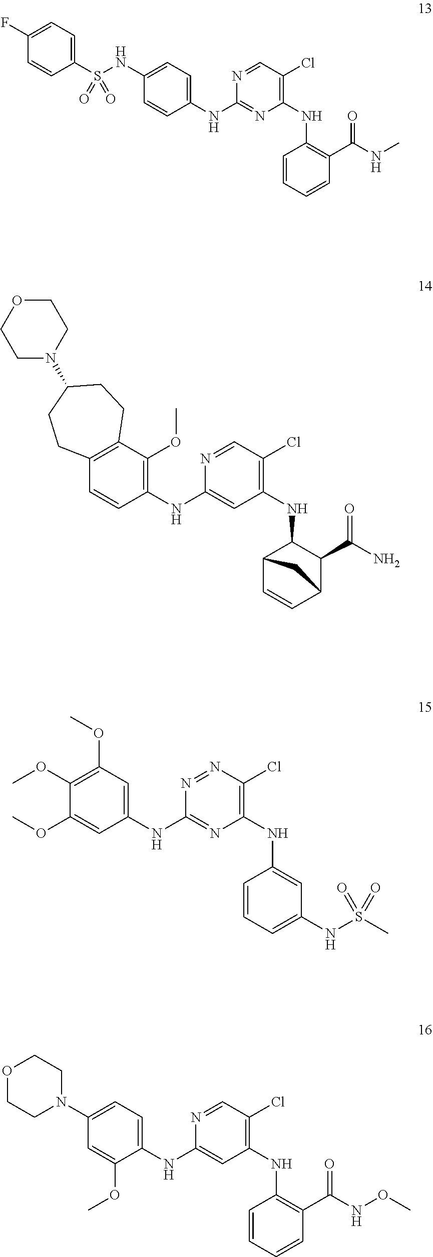 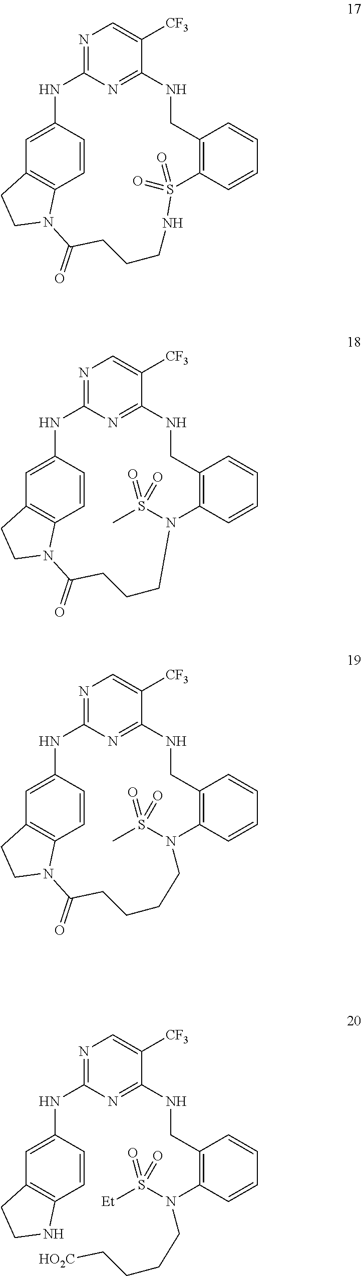 [SMILES: O=C1CCCNS(=O)(=O)c2ccccc2CNc2nc(ncc2C(F)(F)F)Nc2ccc3c(c2)CCN13 ; CS(=O)(=O)N1CCCC(=O)N2CCc3cc(ccc32)Nc2ncc(C(F)(F)F)c(n2)NCc2ccccc21 ; CS(=O)(=O)N1CCCCC(=O)N2CCc3cc(ccc32)Nc2ncc(C(F)(F)F)c(n2)NCc2ccccc21 ; CCS(=O)(=O)N(CCCCC(=O)O)c1ccccc1CNc1nc(Nc2ccc3c(c2)CCN3)ncc1C(F)(F)F] 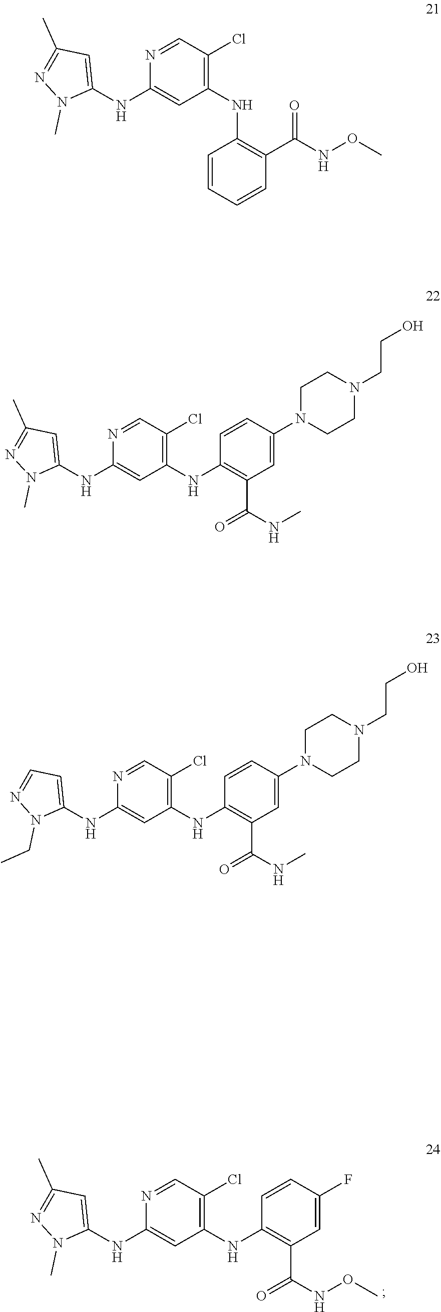
preferably, the FAK inhibitor is the following compounds, or optical isomers thereof, or tautomers thereof, or salts thereof, or prodrugs thereof, or hydrates thereof, or solvates thereof, and the following compounds include 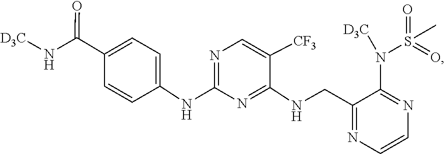 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]
Defactinib or IN10018.

12. The drug combination according to claim 10, characterized in that the CD44 inhibitor is the following compounds, or optical isomers thereof, or tautomers thereof, or salts thereof, or prodrugs thereof, or hydrates thereof, or solvates thereof, or peptides, or antibodies, and the following compounds or peptides or antibodies include Angstrom6, PEP-1, AMC303, Anti-CD44 mAb H4C4, anti-CD44 mAb HI44a, anti-CD44 mAb IM7, anti-CD44 mAb KM201, RO5429083, RG7356, anti-CD44V6 mAb 2F10, anti-CD44V6 mAb U36, anti-CD44V6 mAb V6B3, anti-CD44V6 mAb VFF18, anti-CD44V6 mAb VFF4, anti-CD44V6 and mAb VFF7;
preferably, the CD44 inhibitor is the following compound, or optical isomers thereof, or tautomers thereof, or salts thereof, or prodrugs thereof, or hydrates thereof, or solvates thereof, and the following compound includes AMC303.

13. The drug composition according to claim 10, characterized in that the FAK inhibitor is compound 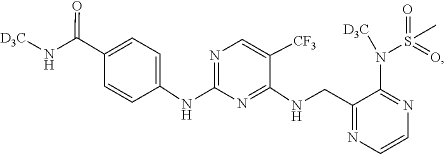 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]
or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-81:1;
alternatively, the FAK inhibitor is Defactinib, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-27:1;
alternatively, the FAK inhibitor is IN10018, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 1:1.

14. The use of FAK inhibitors combined with CD44 inhibitors in the manufacturer of medicaments for treating cancers;
preferably, the weight ratio of FAK inhibitor and CD44 inhibitor is 1:10-10:1;
alternatively, the molar ratio of FAK inhibitor and CD44 inhibitor is 1:82-82:1;
more preferably, the molar ratio of FAK inhibitor and CD44 inhibitor is 0.4-81:1.

15. The use according to claim 14, characterized in that the FAK inhibitors are the following compounds, or optical isomers thereof, or tautomers thereof, or salts thereof, or prodrugs thereof, or hydrates thereof, or solvates thereof, and the following compounds include: Defactinib, CEP-28122, CEP-37440, TAE226, PF-562271, PF-431396, VS-4718, PF-573228, BI853520, IN10018, and APG-2449;
alternatively, the FAK inhibitor is a compound represented by formula I, or optical isomers thereof, or tautomers thereof, or salts thereof, or prodrugs thereof, or hydrates thereof, or solvates thereof, 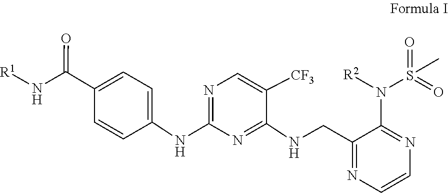
wherein, R¹ and R² are each independently selected from the group consisting of hydrogen, methyl, and trideuteromethyl;
alternatively, the FAK inhibitor is the following compounds, or optical isomers thereof, or tautomers thereof, or salts thereof, or prodrugs thereof, or hydrates thereof, or solvates thereof, and the following compounds are selected from the group consisting of: 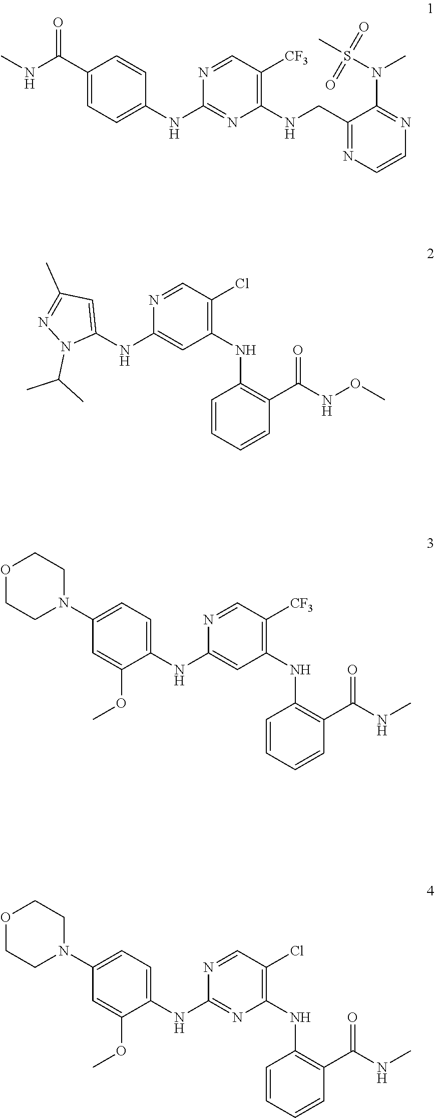 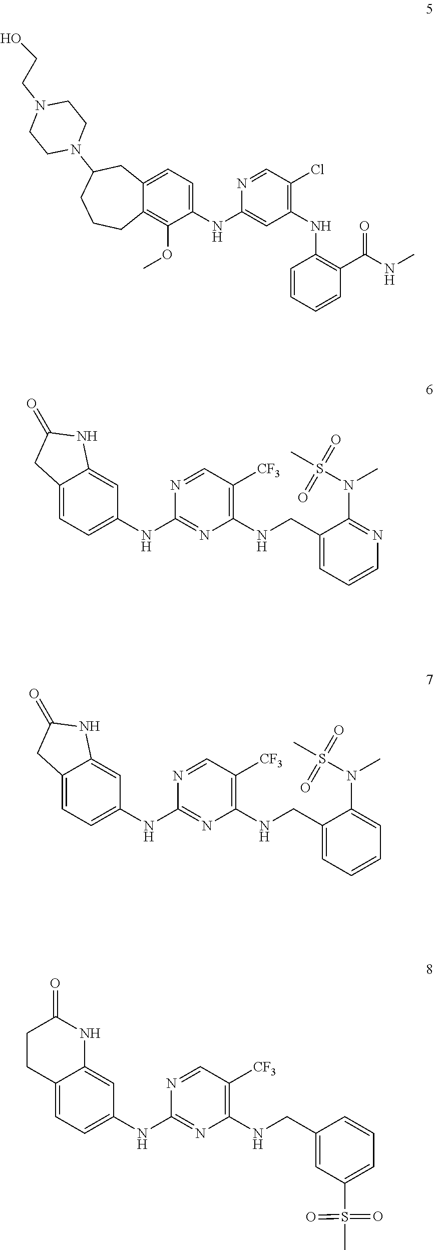 [SMILES: CNC(=O)c1ccccc1Nc1cc(Nc2ccc3c(c2OC)CCCC(N2CCN(CCO)CC2)C3)ncc1Cl ; CN(c1ncccc1CNc1nc(Nc2ccc3c(c2)NC(=O)C3)ncc1C(F)(F)F)S(C)(=O)=O ; CN(c1ccccc1CNc1nc(Nc2ccc3c(c2)NC(=O)C3)ncc1C(F)(F)F)S(C)(=O)=O ; CS(=O)(=O)c1cccc(CNc2nc(Nc3ccc4c(c3)NC(=O)CC4)ncc2C(F)(F)F)c1] 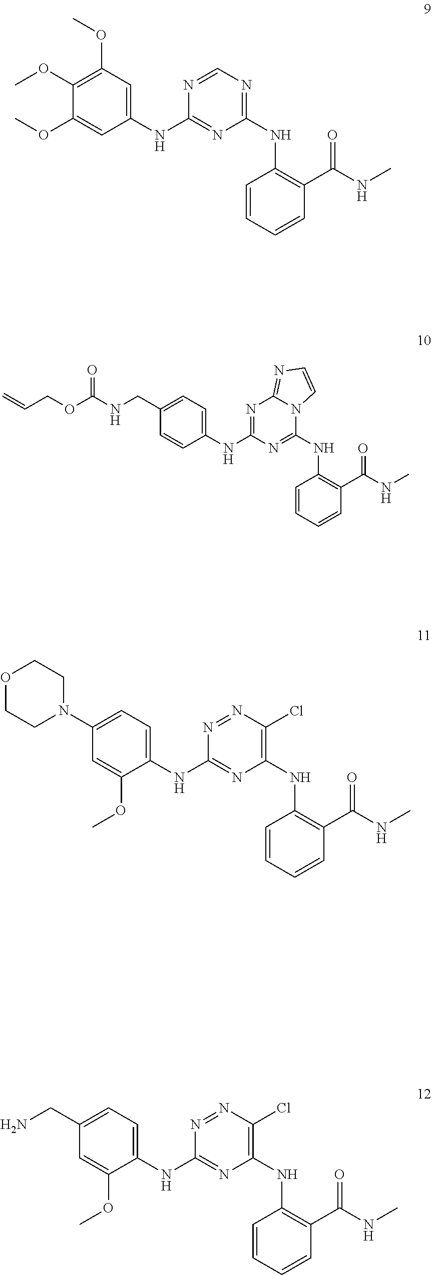 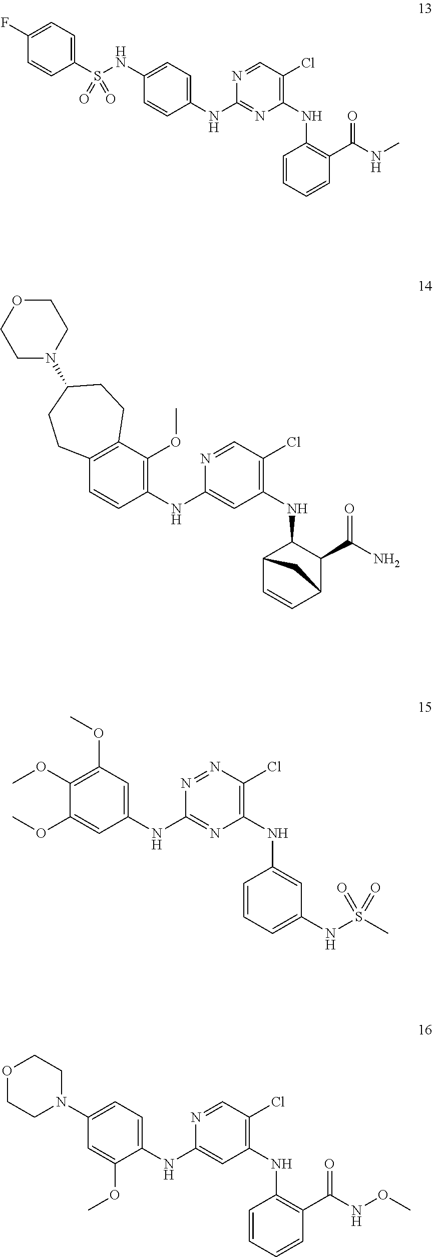 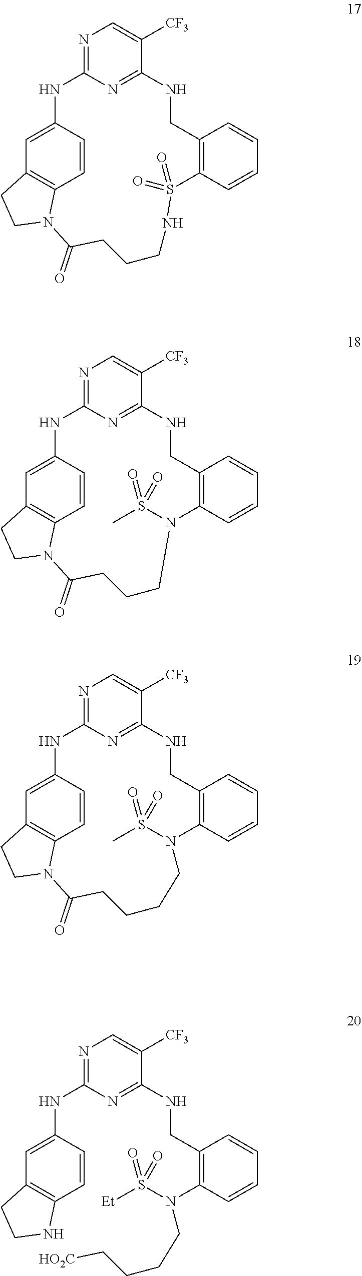 [SMILES: O=C1CCCNS(=O)(=O)c2ccccc2CNc2nc(ncc2C(F)(F)F)Nc2ccc3c(c2)CCN13 ; CS(=O)(=O)N1CCCC(=O)N2CCc3cc(ccc32)Nc2ncc(C(F)(F)F)c(n2)NCc2ccccc21 ; CS(=O)(=O)N1CCCCC(=O)N2CCc3cc(ccc32)Nc2ncc(C(F)(F)F)c(n2)NCc2ccccc21 ; CCS(=O)(=O)N(CCCCC(=O)O)c1ccccc1CNc1nc(Nc2ccc3c(c2)CCN3)ncc1C(F)(F)F] 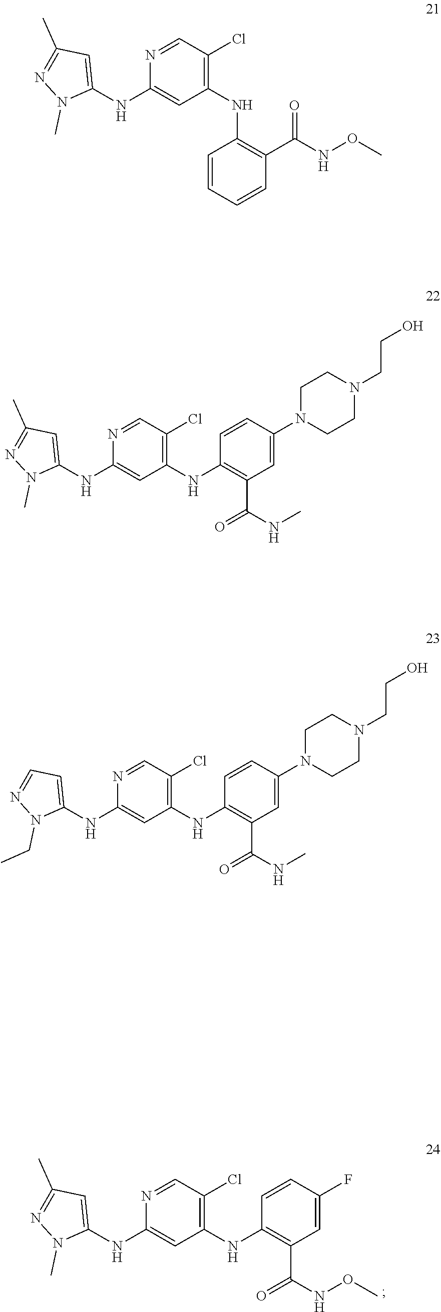
preferably, the FAK inhibitor is the following compounds, or optical isomers thereof, or tautomers thereof, or salts thereof, or prodrugs thereof, or hydrates thereof, or solvates thereof, and the following compounds include 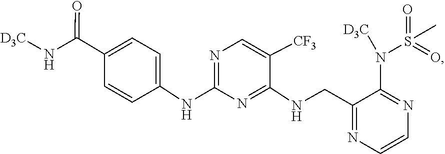 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]
Defactinib or IN10018.

16. The use according to claim 14, characterized in that the CD44 inhibitor is the following compounds, or optical isomers thereof, or tautomers thereof, or salts thereof, or prodrugs thereof, or hydrates thereof, or solvates thereof, or peptides, or antibodies, and the following compounds or peptides or antibodies include Angstrom6, PEP-1, AMC303, Anti-CD44 mAb H4C4, anti-CD44 mAb HI44a, anti-CD44 mAb IM7, anti-CD44 mAb KM201, RO5429083, RG7356, anti-CD44V6 mAb 2F10, anti-CD44V6 mAb U36, anti-CD44V6 mAb V6B3, anti-CD44V6 mAb VFF18, anti-CD44V6 mAb VFF4, anti-CD44V6, and mAb VFF7;
preferably, the CD44 inhibitor is the following compound, or optical isomers thereof, or tautomers thereof, or salts thereof, or prodrugs thereof, or hydrates thereof, or solvates thereof, and the following compound includes AMC303.

17. The use according to claim 14, characterized in that the FAK inhibitor is compound 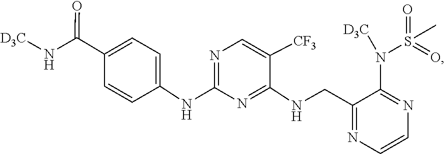 [SMILES: [2H]C([2H])([2H])NC(=O)c1ccc(Nc2ncc(C(F)(F)F)c(NCc3nccnc3N(C([2H])([2H])[2H])S(C)(=O)=O)n2)cc1]
or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-81:1;
alternatively, the FAK inhibitor is Defactinib, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 0.4-27:1;
alternatively, the FAK inhibitor is IN10018, or optical isomers thereof, or tautomers thereof, or salts thereof, or prodrugs thereof, or hydrates thereof, or solvates thereof; the CD44 inhibitor is AMC303, or optical isomers thereof, or tautomers thereof, or salts thereof, or prodrugs thereof, or hydrates thereof, or solvates thereof; the molar ratio of FAK inhibitor and CD44 inhibitor is 1:1.

18. The use according to claim 14, characterized in that the cancer is solid tumor, mesothelioma, melanoma, prostate cancer, breast cancer, glioblastoma, and brain cancer;
the solid tumors include mesothelioma, pancreatic cancer, soft tissue tumor, metastatic tumor, non-solid cancer, sarcoma, adenocarcinoma, lung cancer, breast cancer, lymphoma, gastrointestinal cancer, urogenital system cancer, prostate cancer, ovarian cancer; the gastrointestinal cancer includes colon cancer, and the urogenital system cancer includes renal, urothelial or testicular tumors; the ovarian cancer includes advanced ovarian cancer;
the mesothelioma includes neurofibroma, renal cancer, lung cancer, small cell lung cancer, non-small cell lung cancer, KRAS-mutant non-small cell lung cancer, liver cancer, thyroid cancer, breast cancer, nervous system tumor, neurilemmoma, meningioma, neuroma, adenoid cystic carcinoma, ependymoma, ependymal tumor, malignant pleural tumor, malignant pleural mesothelioma, triplet tumor, negative breast cancer, non-blood malignant tumor, melanoma, colorectal cancer, leukemia, adenocarcinoma, and solid tumors;
the melanoma includes locally advanced melanoma, locally mutant N-Ras driven melanoma, and metastatic malignant skin melanoma; the colorectal cancer includes metastatic colorectal cancer; the leukemia includes acute myelogenous leukemia; the adenocarcinoma includes adenocarcinoma; the solid tumors include locally advanced solid tumors, metastatic solid tumors, and hepatocellular carcinoma;
the prostate cancer includes castration-resistant prostate cancer and metastatic castration resistant prostate cancer;
the brain cancer includes neuroepithelial tissue tumors, cerebral gliomas, astrocytomas, oligodendrogliomas, ependymal and choroidal plexus tumors, pineal tumors, neurocellular tumors, gangliocytoma, neuroblastoma, poorly differentiated tumors, embryonal tumors, glioblastoma multiform, medulloblastoma, schwannoma, meningioma, malignant lymphoma, cerebrovascular tumors, vascular malformations capillary telangiectases, pituitary tumors, and metastatic tumors.
